(19) United States
(12) Patent Application Publication
(10) Pub. No.: US 2015/0057562 A1
(43) Pub. Date: Feb. 26, 2015

(54) APPARATUS FOR MEASURING TEMPERATURE DISTRIBUTION ACROSS THE SOLE OF THE FOOT

(71) Applicant: Podimetrics, Inc., Cambridge, MA (US)

(72) Inventors: David Robert Linders, Somerville, MA (US); Brian Petersen, Somerville, MA (US)

(21) Appl. No.: 14/468,909

(22) Filed: Aug. 26, 2014

Related U.S. Application Data
(60) Provisional application No. 61/869,990, filed on Aug. 26, 2013.

(51) Int. Cl. A61B 5/01; G01K 7/16; A61B 5/00

(52) U.S. Cl. CPC A61B 5/015; A61B 5/6829; G01K 7/16
USPC 600/549

(57) ABSTRACT

An apparatus for measuring the temperature distribution over the sole of the foot has a flexible substrate. The substrate defines a plurality of discontinuities forming a plurality of substrate segments. Each substrate segment has a sensor region with a surface for coupling a sensor, and at least one connector to connect to at least one sensor region of an adjacent substrate segment. Each of a plurality of the sensor regions is configured to be movable relative to other sensor regions. The discontinuities cause the flexible substrate to exhibit elastic properties in the aggregate. The apparatus also has a plurality of resistive contact temperature sensors coupled with the flexible substrate.

### PRIORITY

This patent application claims priority from provisional U.S. patent application No. 61/869,990 filed Aug. 26, 2013, entitled, “SENSOR MATRIX FOR MEASURING PROPERTIES OF A THREE-DIMENSIONAL BODY,” and naming David Robert Linders as inventor, the disclosure of which is incorporated herein, in its entirety, by reference.

### RELATED APPLICATIONS

This patent application is related to the following utility patent applications, each of which is incorporated herein, in its entirety, by reference:

1. U.S. patent application Ser. No. 13/799,828, filed on Mar. 13, 2013, entitled, “METHOD AND APPARATUS FOR INDICATING THE RISK OF AN EMERGING ULCER,” assigned attorney docket number 3891/1001, and naming Jonathan David Bloom, David Robert Linders, Jeffrey Mark Engler, Brian Petersen, Adam Geboff, AND David Charles Kale, and as inventors,

2. U.S. patent application Ser. No. 13/803,866, filed on Mar. 14, 2013, entitled, “METHOD AND APPARATUS FOR INDICATING THE EMERGENCE OF A PRE-ULCER AND ITS PROGRESSION,” assigned attorney docket number 3891/1002, and naming Jonathan David Bloom, David Robert Linders, Jeffrey Mark Engler, Brian Petersen, David Charles Kale, and Adam Geboff as inventors, and

3. U.S. patent application Ser. No. 13/799,847 filed on Mar. 13, 2013, entitled, “METHOD AND APPARATUS FOR INDICATING THE EMERGENCE OF AN ULCER,” assigned attorney docket number 3891/1003, and naming Jonathan David Bloom, David Robert Linders, Jeffrey Mark Engler, Brian Petersen, David Charles Kale, and Adam Geboff as inventors.

### FIELD OF THE INVENTION

The invention generally relates to maintaining foot health and, more particularly, the invention relates to an array of sensors for measuring a physical property of a human foot.

### BACKGROUND OF THE INVENTION

Open sores on an external surface of the body often form septic breeding grounds for infection, which can lead to serious health complications. For example, foot ulcers on the bottom of a diabetic's foot can lead to gangrene, leg amputation, or, in extreme cases, death. The healthcare establishment therefore recommends monitoring a diabetic's foot on a regular basis to avoid these and other dangerous consequences. Unfortunately, known techniques for monitoring foot ulcers, among other types of ulcers, often are inconvenient to use, unreliable, or inaccurate, thus reducing compliance by the very patient populations that need it the most.

### SUMMARY OF VARIOUS EMBODIMENTS

In accordance with one embodiment of the invention, an apparatus for measuring the temperature distribution over the sole of the foot has a flexible substrate formed at least in part from a substrate material configured to be bendable without breaking when receiving the sole of a human foot. The substrate defines a plurality of discontinuities forming a plurality of substrate segments. Each substrate segment has a sensor region with a surface for coupling a sensor, and at least one connector to connect to at least one sensor region of an adjacent substrate segment. Each of a plurality of the sensor regions is configured to be movable relative to other sensor regions when subjected to a mechanical force applied to its surface. The discontinuities cause the flexible substrate to exhibit elastic properties in the aggregate so that it can recover its size and shape after deformation along one or more of the discontinuities.

The apparatus also has a plurality of resistive temperature sensors coupled with the flexible substrate. The plurality of resistive temperature sensors preferably are contact sensors for measuring the temperature of a foot in thermal contact with it. A plurality of the sensor regions of the substrate segments each are coupled with at least one of the plurality of temperature sensors. In addition, the apparatus also has a matrix of circuit conductors extending across the flexible substrate (e.g., on a surface or internal to the substrate) to electrically interconnect the plurality of resistive contact temperature sensors. A plurality of the connectors each physically bridges a portion of the matrix of conductors between sensor regions of adjacent substrate segments to electrically connect adjacent temperature sensors. At least some of the substrate segments and matrix of circuit conductors are configured to permit relative substrate segment movement without mechanically breaking the portion of the matrix of circuit conductors on the connector between substrate segments moving relatively to each other.

The flexible substrate may be formed at least in part from an inelastic substrate material, or an elastic material. In addition or alternatively, the substrate forms an open platform or a closed platform. The plurality of connectors may have a variety of shapes, such as a serpentine shape. Moreover, the plurality of the discontinuities can form an opening between adjacent substrate segments. In contrast, at least two adjacent substrate segments can abut each other.

Some embodiments of the substrate material include circuit board material. For example, the substrate may include a flexible polymeric circuit material (e.g., a flexible circuit, sometimes known as “Flex”), such as polyimide or polyester. Alternatively, the substrate may include a traditional circuit material, such as FR-4 configured to a thickness that provides flexible properties to the substrate. Each of a plurality of the sensor regions may be configured to be movable relative to other sensor regions when subjected to a mechanical force applied generally normal to its surface. To improve certain performance, the plurality of resistive temperature sensors may be non-uniformly spaced across the flexible substrate.

The apparatus also may have logic for determining either the risk of an ulcer forming on the foot, or the presence of an ulcer on the foot. In addition or alternatively, the apparatus also may have logic for measuring the temperature distribution across the foot and forming a thermogram of the temperature distribution.

In accordance with another embodiment, an apparatus for analyzing the temperature distribution over the sole of the human foot has an open platform with a flexible substrate formed at least in part from a substrate material configured to be bendable without breaking when receiving the sole of a human foot. The substrate material is inelastic, and the substrate forms a plurality of substrate segments. Each substrate segment has a sensor region for coupling a sensor, and at least one connector to connect to at least one sensor region of an adjacent substrate segment. Each of a plurality of the sensor regions is configured to be movable relative to other sensor regions when subjected to a mechanical force applied to its surface. Moreover, the discontinuities cause the flexible substrate to exhibit elastic properties so that it can recover its size and shape after deformation along one or more of the discontinuities.

In a manner similar to some other embodiments, this embodiment also has a plurality of resistive temperature sensors coupled with the flexible substrate. A plurality of the sensor regions of the substrate segments are each coupled with at least one of the plurality of temperature sensors. Circuit conductors extend across the flexible substrate to electrically interconnect the plurality of resistive temperature sensors. A plurality of the connectors each physically bridges a portion of the conductors between sensor regions of adjacent substrate segments to electrically connect adjacent temperature sensors. At least some of the substrate segments and circuit conductors are configured to permit relative substrate segment movement without mechanically breaking the portion of the circuit conductors on the connector between substrate segments moving relatively to each other.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a foot having a prominent foot ulcer and a pre-ulcer.

FIG. 2A schematically shows one use and form factor that may be implemented in accordance with illustrative embodiments of the invention.

FIG. 2B schematically shows an open platform that may be configured in accordance with illustrative embodiments of the invention.

FIG. 3 schematically shows a cross-sectional view of a human foot on the open platform of FIG. 2B. This figure just shows a top layer of one embodiment of the open platform.

FIG. 4A schematically shows an exploded view of one type of open platform that may be configured in accordance with illustrative embodiments of the invention.

FIG. 4B schematically shows a close-up view of the platform with details of the pads and temperature sensors.

FIG. 5A schematically shows a cross-sectional view of a substrate and its requisite components configured in accordance with illustrative embodiments of the invention.

FIGS. 5B and 5C respectively show top and bottom views of the substrate of FIG. 5A.

FIG. 6A schematically shows one embodiment of a substrate that may be used in accordance with illustrative embodiments of the invention.

FIG. 6B schematically shows a close up view of the embodiment of FIG. 6A.

FIG. 7A schematically shows another embodiment of a substrate that may be used in accordance with illustrative embodiments of the invention.

FIG. 7B schematically shows a close up view of the embodiment of FIG. 7A.

FIGS. 8A and 8B schematically show two alternative arrangements of circuit traces and sensors on the substrate of FIGS. 7A and 7B.

FIG. 9A schematically shows a perspective view of the substrate of FIG. 7A with no applied load.

FIGS. 9B and 9C schematically show perspective and cross-sectional views, respectively, of the substrate of FIG. 7A with a load applied to one portion.

FIGS. 9D and 9E schematically show perspective and cross-sectional views, respectively, of the substrate of FIG. 7A with a larger load applied to one portion.

FIGS. 10A-10D schematically show views of yet other substrate designs that may be produced in accordance with illustrative embodiments.

FIGS. 11 and 12 schematically show top views of two illustrative non-uniform sensor layouts across a substrate.

FIG. 13 schematically shows a sensor array implementing illustrative embodiments of the invention.

FIG. 14 shows a control logic table for the sensor array of FIG. 13 in accordance with illustrative embodiments of the invention.

FIG. 15 shows a process of monitoring the health of the patient's foot or feet in accordance with illustrative embodiments the invention.

FIG. 16 shows a process of forming a thermogram in accordance with illustrative embodiments of the invention.

FIGS. 17A-17D schematically show the progression of the thermogram and how it is processed in accordance with one embodiment of the invention.

FIGS. 18A and 18B schematically show two different types of patterns that may be on the soles of a patient's foot indicating an ulcer or pre-ulcer.

FIGS. 19A and 19B schematically show two different user interfaces that may be displayed in accordance with illustrative embodiments of the invention.

### DESCRIPTION OF ILLUSTRATIVE EMBODIMENTS

In illustrative embodiments, an apparatus or platform uses contact sensors to more completely and easily measure the temperature distribution across the three-dimensional landscape of the sole of a foot. To that end, the apparatus or platform has a flexible substrate with substrate segments that more readily conform to the shape of the sole of the foot. In effect, even when formed from a material that is inelastic, the flexible substrate in the aggregate has elastic qualities, enabling it to conform to the foot. Accordingly, surfaces of the sole that are not necessarily flat (e.g., a foot surface that is nearly normal to the floor when standing) can be appropriately analyzed for disease, such as the formation of ulcers or pre-ulcers. Details of illustrative embodiments are discussed below.

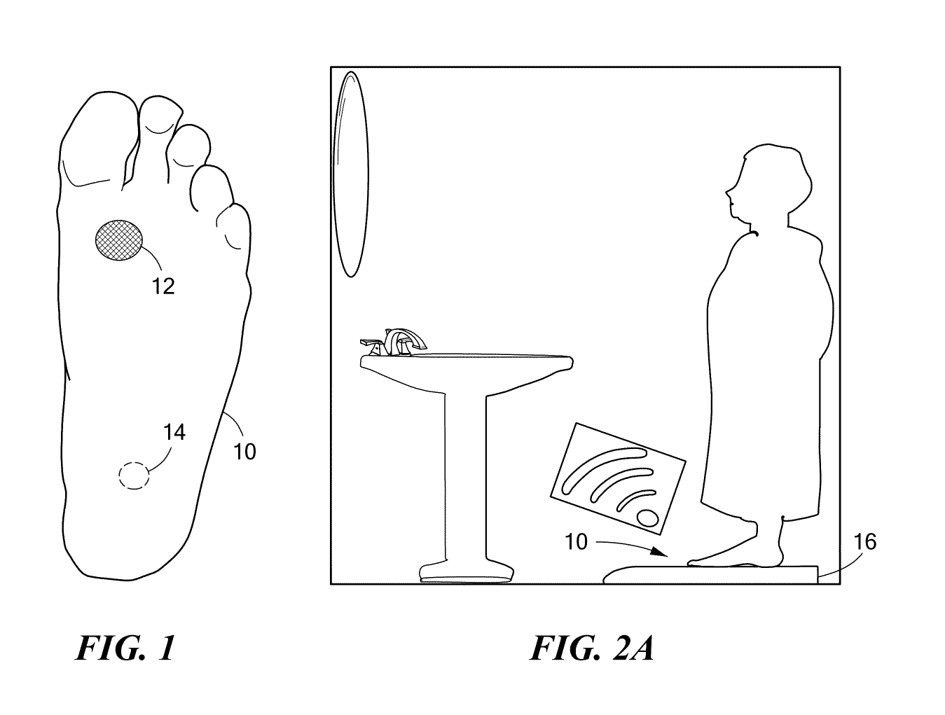

FIG. 1 schematically shows a bottom view of a patient's foot 10 that, undesirably, has an ulcer 12 and a pre-ulcer 14 (described below and shown in phantom since pre-ulcers 14 do not break through the skin). As one would expect, an ulcer 12 on this part of the foot 10 typically is referred to as a “foot ulcer 12.” Generally speaking, an ulcer is an open sore on a surface of the body generally caused by a breakdown in the skin or mucous membrane. Diabetics often develop foot ulcers 12 on the soles of their feet 10 as part of their disease. In this setting, foot ulcers 12 often begin as a localized inflammation that may progress to skin breakdown and infection.

It should be noted that discussion of diabetes and diabetics is but one example and used here simply for illustrative purposes only. Accordingly, various embodiments apply to other types of diseases (e.g., stroke, deconditioning, sepsis, friction, coma, etc. . . . ) and other types of ulcers—such embodiments may apply generally where there is a compression or friction on the living being's body over an extended period of time. For example, various embodiments also apply to ulcers formed on different parts of the body, such as on the back (e.g., bedsores), inside of prosthetic sockets, or on the buttocks (e.g., a patient in a wheel chair). Moreover, alternative embodiments apply to other types of living beings beyond human beings, such as other mammals (e.g., horses or dogs). Accordingly, discussion of diabetic human patients having foot ulcers 12 is for simplicity only and not intended to limit all embodiments of the invention.

Many prior art ulcer detection technologies known to the inventors suffered from one significant problem—patient compliance. If a diseased or susceptible patient does not regularly check his/her feet 10, then that person may not learn of an ulcer 12 or a pre-ulcer 14 until it has emerged through the skin and/or requires significant medical treatment. Accordingly, illustrative embodiments implement an ulcer monitoring system in any of a variety of forms—preferably in an easy to use form factor that facilitates and encourages regular use.

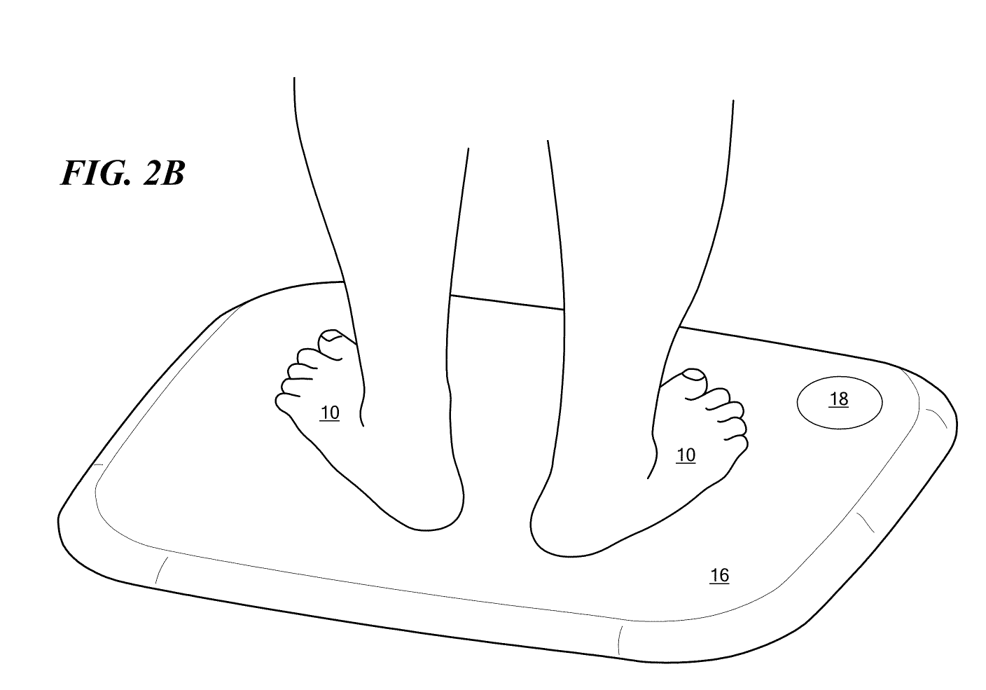

FIGS. 2A and 2B schematically show one form factor, in which a patient/user steps on an open platform 16 that gathers data about that user's feet 10. In this particular example, the open platform 16 is in the form of a floor mat placed in a location where the patient regularly stands, such as in front of a bathroom sink, next to a bed, in front of a shower, on a footrest, or integrated into a mattress. As an open platform 16, the patient simply may step on the top sensing surface of the platform 16 to initiate the process and then step off. There is no need to fit inside something. Accordingly, this and other form factors favorably do not require that the patient affirmatively decide to interact with the platform 16. Instead, many expected form factors are configured to be used in areas where the patient frequently stands during the course of their day without a foot covering. Alternatively, the open platform 16 may be moved to directly contact the feet 10 of a patient that cannot stand. For example, if the patient is bedridden, then the platform 16 may be brought into contact with the patient's feet 10 while in bed.

A bathroom mat or rug are but two of a wide variety of different potential form factors. Others may include a platform 16 resembling a scale, a stand, a footrest, a console, a tile built into the floor, or a more portable mechanism that receives at least one of the feet 10. The implementation shown in FIGS. 2A and 2B has a top surface area that is larger than the surface area of one or both of the feet 10 of the patient. This enables a caregiver to obtain a complete view of the patient's entire sole, providing a more complete view of the foot 10.

The open platform 16 also has some indicia or display 18 on its top surface they can have any of a number of functions. For example, the indicia can turn a different color or sound an alarm after the readings are complete, show the progression of the process, or display results of the process. Of course, the indicia or display 18 can be at any location other than on the top surface of the open platform 16, such as on the side, or a separate component that communicates with the open platform 16. In fact, in addition to, or instead of, using visual or audible indicia, the platform 16 may have other types of indicia, such as tactile indicia/feedback, our thermal indicia.

Rather than using an open platform 16, alternative embodiments may be implemented as a closed platform 16, such as a shoe or sock that can be regularly worn by a patient, or worn on an as-needed basis. For example, the insole of the patient's shoe or boot may have the functionality for detecting the emergence of a pre-ulcer 14 or ulcer 12, and/or monitoring a pre-ulcer 14 or ulcer 12.

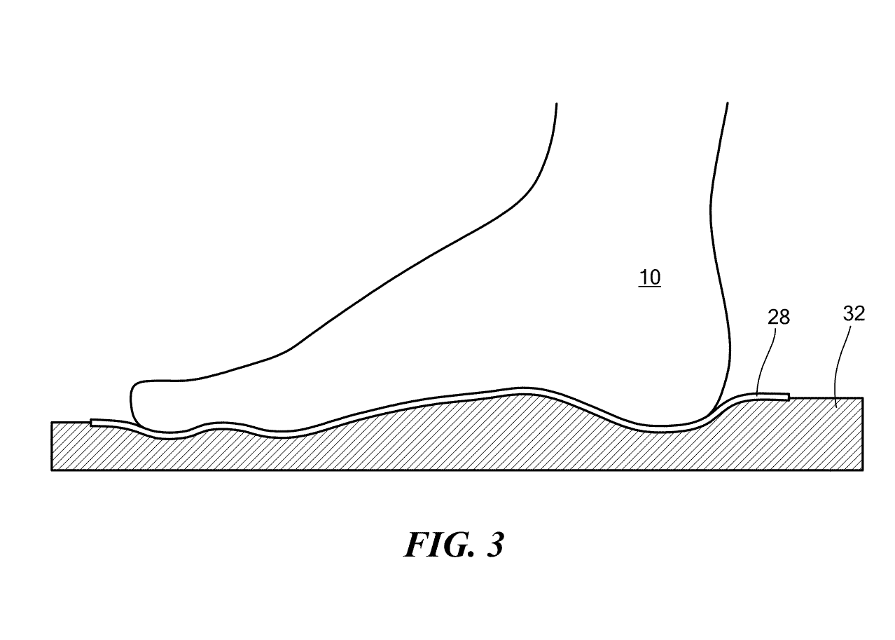

FIG. 3 schematically shows a side, cross-sectional view of a top part of the open platform 16 of FIGS. 2A and 2B. As shown, the top part may include foam or other material that conforms to the sole of the foot 10. The substrate, discussed below, should conform in a similar manner.

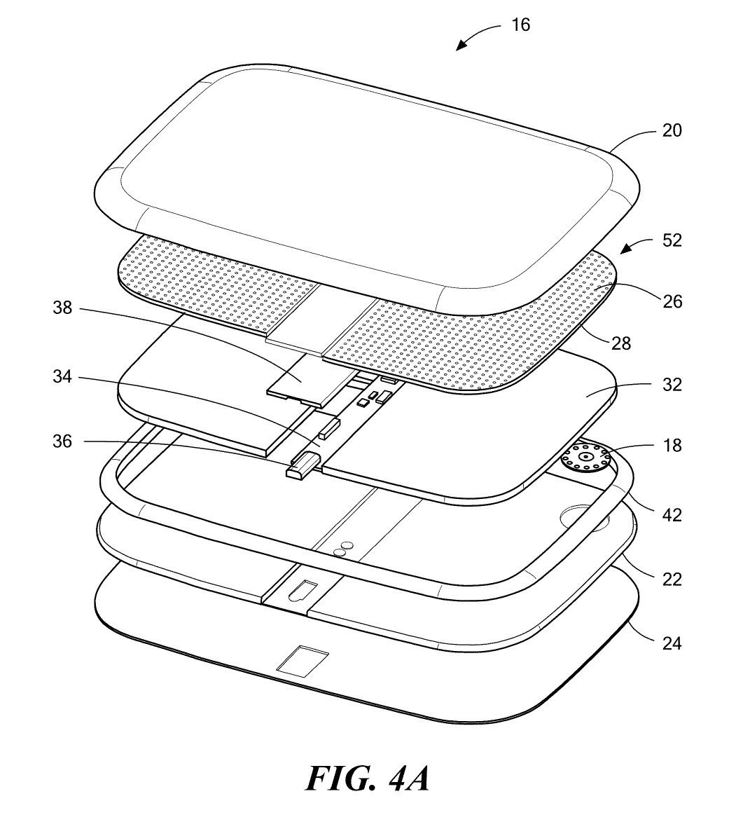

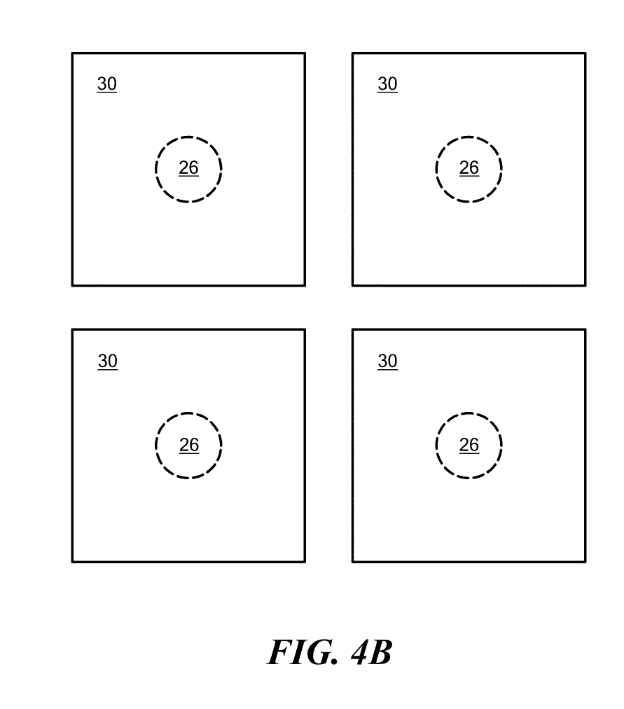

To monitor the health of the patient's foot 10 (discussed in greater detail below), the platform 16 of FIGS. 2A and 2B gathers temperature data about a plurality of different locations on the sole of the foot 10. This temperature data provides the core information ultimately used to determine the health of the foot 10. FIG. 4A schematically shows an exploded view of the open platform 16 configured and arranged in accordance with one embodiment of the invention. Of course, this embodiment is but one of a number of potential implementations and, like other features, is discussed by example only.

As shown, the platform 16 is formed as a stack of functional layers sandwiched between a cover 20 and a rigid base 22. For safety purposes, the base 22 preferably has rubberized or has other non-skid features on its bottom side. FIG. 3 shows one embodiment of this non-skid feature as a non-skid base 24. The platform 16 preferably has relatively thin profile to avoid tripping the patient and making it easy to use.

To measure foot temperature, the platform 16 has an array or matrix of temperature sensors 26 fixed in place directly underneath the cover 20. More specifically, the temperature sensors 26 are positioned on a relatively large printed circuit board/substrate (referred to herein as “printed circuit board 28” or “substrate 28”). The sensors 26 preferably are laid out in a two-dimensional array/matrix on the printed circuit board 28. The pitch or distance between the sensors preferably is relatively small (if uniformly spaced apart or not), thus permitting more temperature sensors 26 on the array. Among other things, the temperature sensors 26 may include temperature sensitive resistors (e.g., printed or discrete components mounted onto the circuit board 28), thermocouples, fiber optic temperature sensors, or a thermochromic film. Accordingly, when used with temperature sensors 26 that require direct contact, illustrative embodiments form the cover 20 with a thin material having a relatively high thermal conductivity. The platform 16 also may use temperature sensors 26 that can still detect temperature through a patient's socks.

Other embodiments may use noncontact temperature sensors 26, such as infrared detectors. Indeed, in that case, the cover 20 may have openings to provide a line of sight from the sensors 26 to the sole of the foot 10. Accordingly, discussion of contact sensors is by example only and not intended to limit various embodiments. As discussed in greater detail below and noted above, regardless of their specific type, the plurality of sensors 26 generate a plurality of corresponding temperature data values for a plurality of portions/spots on the patient's foot 10 to monitor the health of the foot 10.

Some embodiments also may use pressure sensors for various functions, such as to determine the orientation of the feet 10 and/or to automatically begin the measurement process. Among other things, the pressure sensors may include piezoelectric, resistive, capacitive, or fiber-optic pressure sensors. This layer of the platform 16 also may have additional sensor modalities beyond temperature sensors 26 and pressure sensors, such as positioning sensors, GPS sensors, accelerometers, gyroscopes, and others known by those skilled in the art.

To reduce the time required to sense the temperature at specific points, illustrative embodiments optionally position an array of heat conducting pads 30 over the array of temperature sensors 26. To illustrate this, FIG. 4B schematically shows a small portion of the array of temperature sensors 26 showing four temperature sensors 26 and their pads 30. The temperature sensors 26 are drawn in phantom because they preferably are covered by the pads 30. Some embodiments do not cover the sensors 26, however, and simply thermally connect the sensors 26 with the pads 30.

Accordingly, each temperature sensor 26 has an associated heat conducting pad 30 that channels heat from one two dimensional portion of the foot 10 (considered a two dimensional area although the foot 10 may have some depth dimensionality) directly to its exposed surface. In some embodiments, the array of conducting pads 30 preferably takes up the substantial majority of the total surface area of the printed circuit board 28. The distance between the pads 30 thermally isolates them from one another, thus eliminating thermal short-circuits.

For example, each pad 30 may have a square shape with each side having a length of between about 0.1 and 1.0 inches. The pitch between pads 30 thus is less than that amount. Accordingly, as a further detailed example, some embodiments may space the temperature sensors 26 about 0.4 inches apart with 0.25 inch (per side) square pads 30 oriented so that each sensor 26 is at the center of the square pads 30. This leaves an open region (i.e., a pitch) of about 0.15 inches between the square pads 30. Among other things, the pads 30 may be formed from a film of thermally conductive metal, such as a copper.

As suggested above, some embodiments do not use an array of temperature sensors 26. Instead, such embodiments may use a single temperature sensor 26 that can obtain a temperature reading of most or all of the sole. For example, a single sheet of a heat reactive material, such as a thermochromic film (noted above), or similar apparatus should suffice. As known by those in the art, a thermochromic film, based on liquid crystal technology, has internal liquid crystals that reorient to produce an apparent change in color in response to a temperature change, typically above the ambient temperature. This film may serve the role of the substrate 28. Alternatively, one or more individual temperature sensors 26, such as thermocouples or temperature sensor resistors, may be movable to take repeated temperature readings across the bottom of the foot 10.

To operate efficiently, the open platform 16 should be configured so that its top surface contacts substantially the entire sole of the patient's foot 10. To that end, the platform 16 optionally has a flexible and movable layer of foam 32, noted above, or other material that conforms to the user's foot 10. For example, this layer should conform to the arch of the foot 10.

Of course, the printed circuit board 28, and cover 20 also should be similarly flexible and yet robust to conform to the foot 10 in a corresponding manner. Accordingly, the printed circuit board 28 preferably is formed largely from a flexible material that supports the circuit. For example, the printed circuit board 28 may be formed primarily from a flex circuit that supports the temperature sensors 26, or it may be formed from strips of material that individually flex when receiving feet. Details of the substrate/circuit board 28 are discussed below.

The rigid base 22 positioned between the foam 32 and the non-skid base 24 provides rigidity to the overall structure. In addition, the rigid base 22 is contoured to receive a motherboard 34, a battery pack 36, a circuit housing 38, and additional circuit components that provide further functionality. For example, the motherboard 34 may contain integrated circuits and microprocessors that control the functionality of the platform 16.

In addition, the motherboard 34 also may have a user interface/indicia display 18 as discussed above, and a communication interface 40 (not shown) to connect to a larger network 44, such as the Internet. The communication interface 40 may connect wirelessly or through a wired connection with the larger network 44, implementing any of a variety of different data communication protocols, such as Ethernet. Alternatively, the communication interface 40 can communicate through an embedded Bluetooth or other short range wireless radio that communicates with a cellular telephone network 44 (e.g., a 3G or 4G network).

The platform 16 also may have edging 42 and other surface features that improve its aesthetic appearance and feel to the patient. The layers may be secured together using one or more of an adhesive, snaps, nuts, bolts, or other fastening devices.

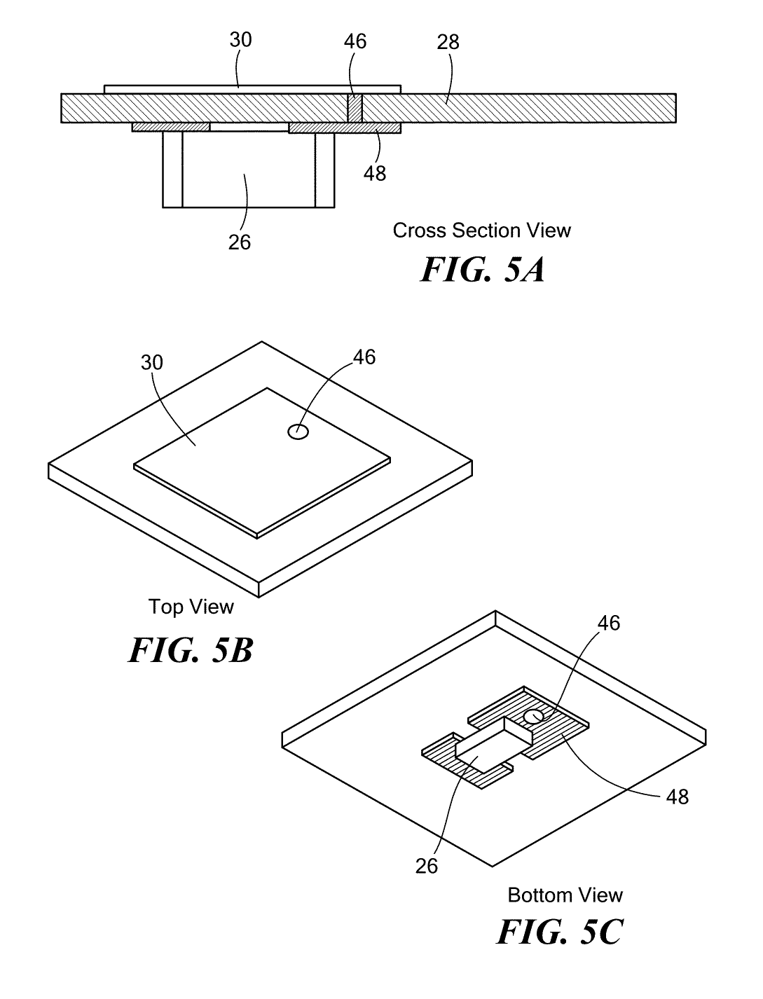

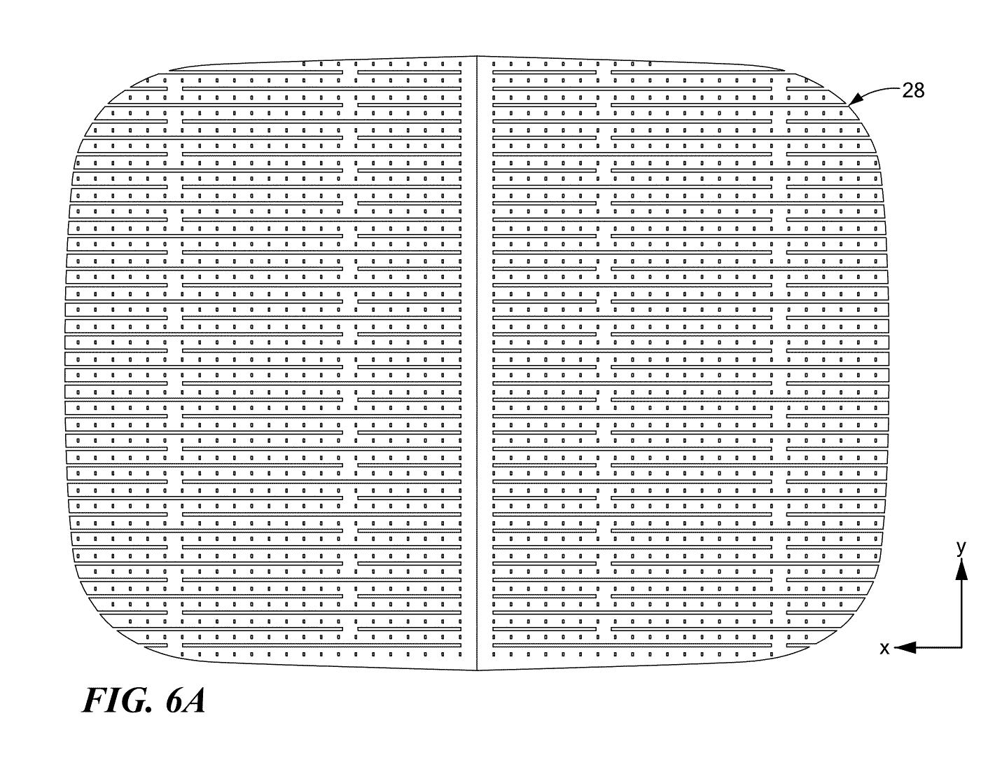

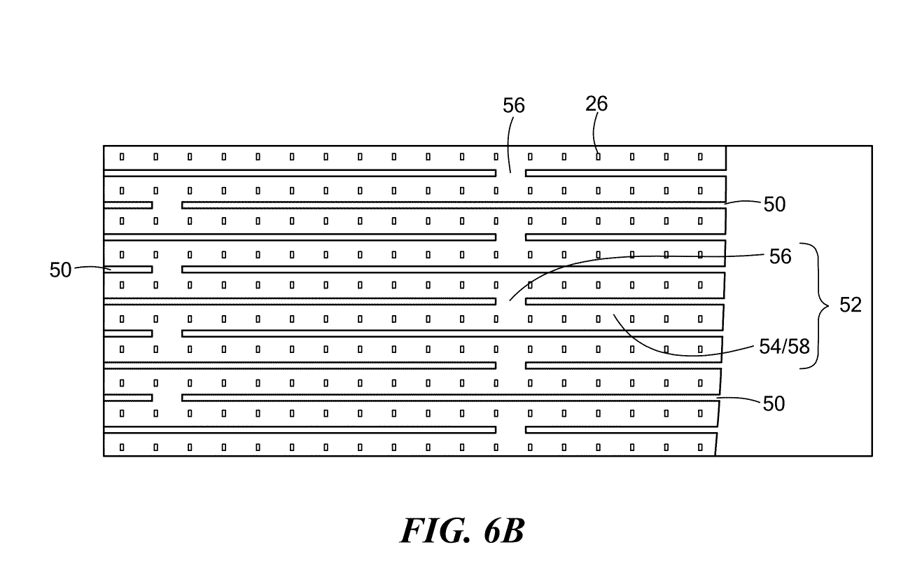

FIG. 5A schematically shows a cross-sectional view of the substrate 28, while FIGS. 5B and 5C respectively show top and bottom perspective use of the a small portion of the substrate 28; namely, a portion of the substrate 28 having only a single temperature sensor 26. As shown, the substrate 28 has the heat conducting pad 30 on its top surface to absorb heat from the foot 10, a resistive, contact temperature sensor 26 on the bottom surface, and a thermally conductive via 46 extending from the top surface and through the substrate 28 to a solder pad 48 to which the temperature sensor 26 is mounted. The bottom surface of the substrate 28 also has at least one other solder pad 48, which can be used to electrically connect the temperature sensor 26 with other devices and sensors (e.g., other temperature sensors 26 on the substrate 28). In illustrative embodiments, the temperature sensor 26 is a surface mounted thermistor. Accordingly, heat collected by the heat conducting pad 30 travels through the via 46 and solder pad 48 to the temperature sensor 26. As discussed below, the temperature sensor 26 uses this temperature data to assess the health of the foot 10.

As noted above, the circuit board 28 (or substrate 28) preferably is fabricated at least in part from a substrate 28 material that is flexible—the material normally is bendable, but will not break when subject to normally expected forces. For example, such expected forces may include a person's foot 10, and the body weight behind that person's foot 10 when someone simply steps onto the platform 16. Indeed, when subjected to extraordinary forces, such as high-G forces (e.g., a strong hammer strike, someone jumping repeatedly, or a gunshot), may break the substrate 28. Illustrative embodiments may form the substrate 28 in part from material that is normally rigid when thick, but flexible when thin (e.g., a 0.01 inch thick sheet of a traditional circuit board material, such as FR-4). As noted above, other embodiments may satisfy this embodiment at least in part using a flexible circuit board. For example, the substrate may include a flexible polymeric circuit material, such as polyimide or polyester.

In addition to being flexible, the substrate material may be inelastic. In other words, the substrate material has the intrinsic quality of normally not recovering its size and shape after being deformed. A substrate 28 that is inelastic, however, may not make direct contact with significant portions of the foot 10 because it may not deform sufficiently when applied to a compound three dimensional surface. A substrate 28 that is elastic favorably may resolve this issue, and yet create more problems. Specifically, the substrate 28 has a number of fragile circuits and conductive connectors (e.g., the circuit traces 60) throughout its body. Many of the circuit traces 60 are printed directly onto the substrate 28. Accordingly, stretching a part of the substrate 28 necessarily may break the circuit traces 60, rendering the platform 16 ineffective.

The inventors discovered that they could resolve this problem by engineering the substrate 28 to exhibit elastic properties in the aggregate while maintaining its local inelasticity. The substrate 28 therefore can flex and effectively stretch to contour with the foot 10, while not locally stretching to break the fragile circuit traces 60.

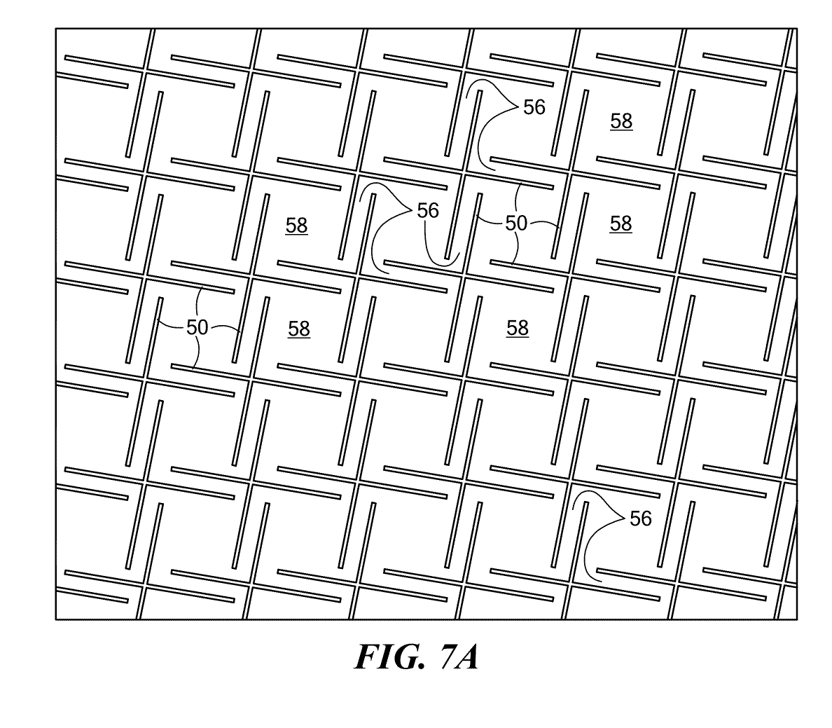

To that end, the substrate 28 has a plurality of discontinuities 50 extending through its body. These discontinuities 50 are considered to divide the substrate 28 into a plurality of substrate segments 52. FIG. 6A schematically shows a plan view of one implementation of this circuit board 28, while FIG. 6B schematically shows a close-up view of the same circuit board 28. The circuit board 28 has two mirror image halves; each half is intended to receive one foot 10. As shown, the circuit board 28 has a plurality of strips 54 running along the x-axis connected together by a plurality of staggered connectors 56, extending between the strips 54, in the y-direction. More specifically, each strip 54 has a sensor region 58 for mounting one or more sensors 26, and one or more shared connectors 56 for connecting with other sensor regions 58.

The strips 54 in this and other embodiments are separated by discontinuities 50 or spaces that enable the sensor regions 58 to move relatively independently of one another. The connectors 56 act as returns or springs that, as discussed in detail below, effectively provide an aggregate elasticity to the substrate 28. When not subjected to a force, the face of the substrate 28 preferably is generally flat, or the various substrate segments 52 generally smoothly transition to adjacent substrate segments 52. For example, the adjacent edges of adjacent substrate segments 52 preferably are aligned to about the same orientation, height, and follow a substantially same contour. Other embodiments, however, may have edges that are not aligned.

The discontinuities 50 thus may form a space between adjacent substrate segments 52/sensor regions 58 when not subjected to a force. It nevertheless should be noted in some embodiments, the edges of the substrate segments 52/sensor regions 58 may abut adjacent substrate segments 52/sensor regions 58. In fact, a single substrate 28 may have some abutting substrate segments 52, and other non-abutting substrate segments 52.

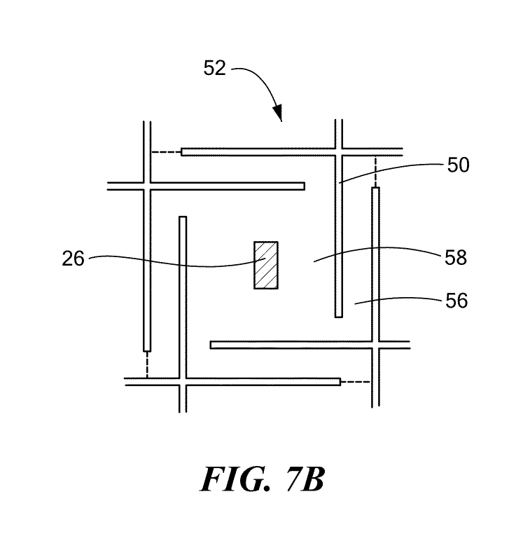

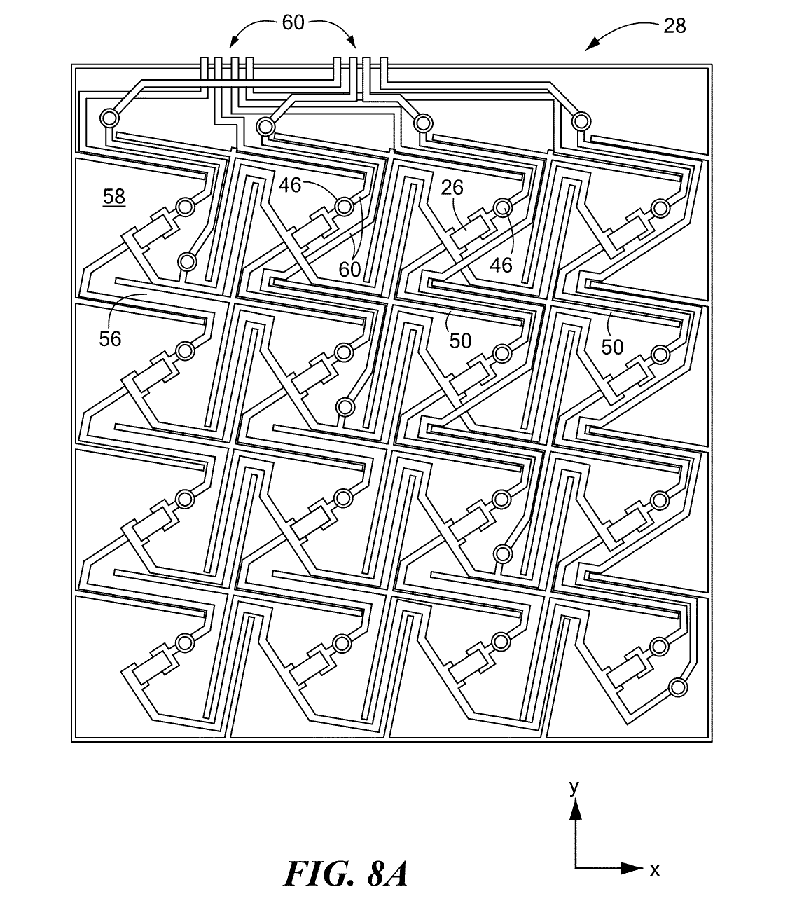

The embodiment of FIGS. 6A and 6B show longitudinal strips 58 connected by short connectors/springs 56. FIG. 7A schematically shows a plan view of another embodiment of the substrate 28, and which the crosses/lines represent discontinuities 50 or cuts 50 made to the substrate 28. The large square areas thus act as the sensor regions 58, while the areas between the lines effectively form connectors 56 between other substrate segments 52. FIG. 7B schematically shows a close-up plan view of the substrate segments 52 of FIG. 7B. In addition, FIG. 7B also schematically shows a sensor 26 mounted to the sensor region 58 of the shown substrate segment 52.

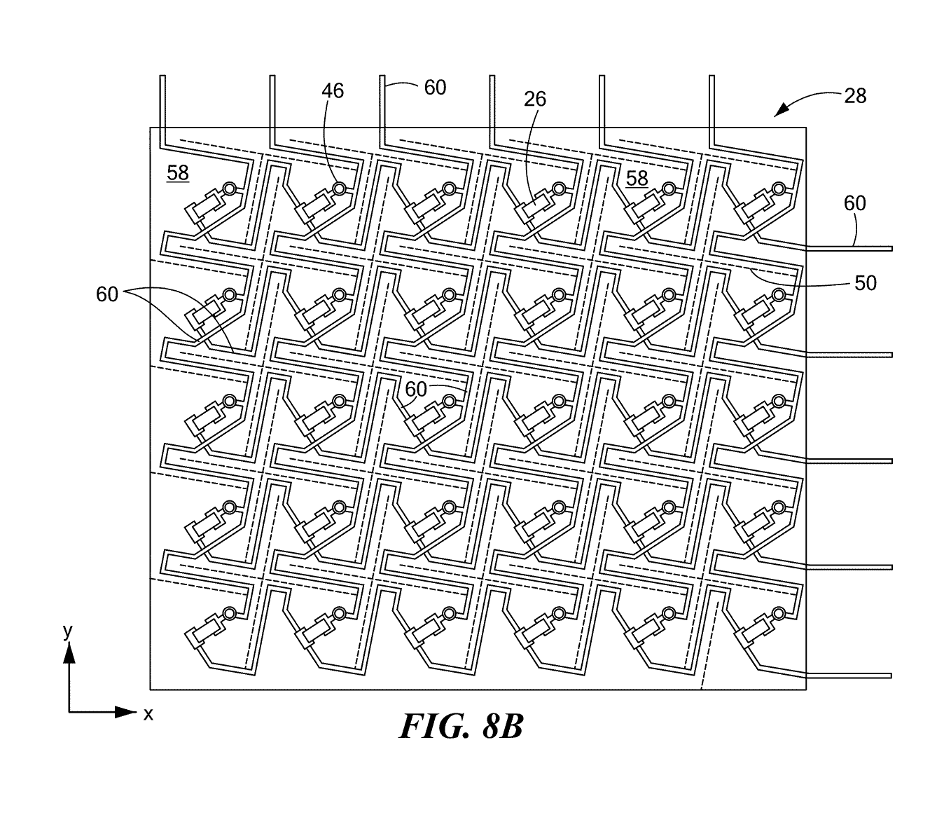

FIG. 8A schematically shows a plan view of the layout of the substrate 28 in accordance with illustrative embodiments of the invention. As shown, this layout has a plurality of discontinuities 50 that effectively form the substrate segments 52. Each substrate segment 52 has a sensor 26 with a conductive via 46 for connecting with the above noted pad 30, and a matrix of conductive circuit traces 60 (e.g., formed from metal, such as copper) connecting its temperature sensor 26 to devices on the substrate 28, and exterior to the substrate 28. For example, from the perspective of the drawing, all of the circuit traces 60 terminate at the top of the substrate 28. As discussed below, to effectively determine which sensor 26 is detecting an elevated temperature, the traces 60 may be considered to form “row” traces 60 that generally extend along the x-axis (or a small angle relative to the x-axis), and “column” traces 60 that generally extend along the y-axis (or a small angle relative to the y-axis). This common termination at the top should simplify the overall platform design by effectively acting as a single, localized interface point. This is in contrast to a similar design of FIG. 8B, in which one set of traces 60 terminates at the top of the substrate 28, and the other set of traces 60 terminates at the right side of the substrate 28.

Those skilled in the art can determine the layer of the substrate 28 to carry the traces 60 and the sensors 26. For example, the temperature sensors 26 can be positioned on the bottom surface of the substrate 28. In a similar manner, the row and column traces 60 also may extend along the bottom surface. Other embodiments, however, may extend the row traces 60 along one surface (e.g., along the bottom surface), and the column traces 60 along another surface (e.g., along the top surface). In fact, some embodiments can embed the traces 60, sensors 26, and/or other elements within the substrate 28.

Connectors 56 between adjacent sensor regions 58 of various embodiments physically bridge or support the traces 60 to form an unbroken circuit across the matrix. In other words, these traces 60 extend between (and past, in many cases) sensors 26 in adjacent sensor regions 58. As noted above, however, the connectors 56 flex, but do not appreciably stretch, when subjected to anticipated forces. The traces 60 thus are anticipated to withstand such flexing forces, maintaining their structural integrity. Testing of similar designs has proven the noted trace robustness when subjected to such forces. Indeed, the traces 60 may break when subjected to extraordinary use, such as scraping against a hard object, or other unintended activities.

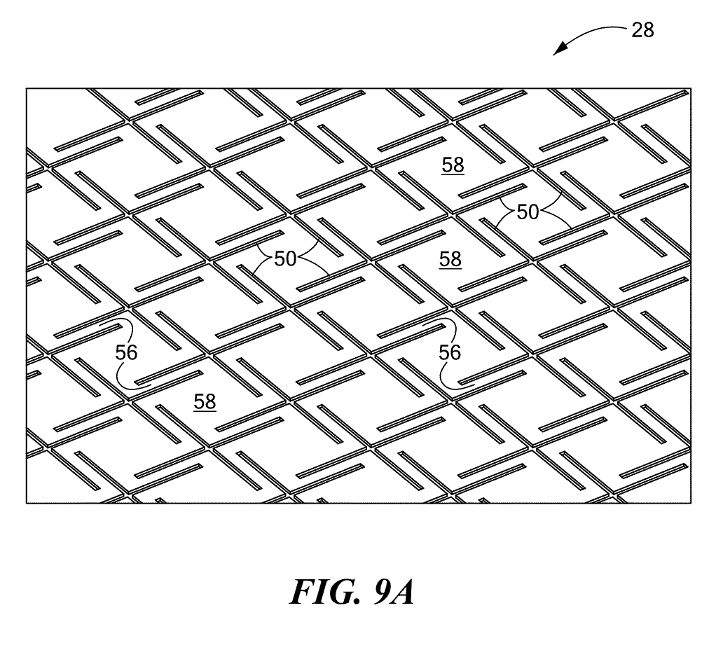

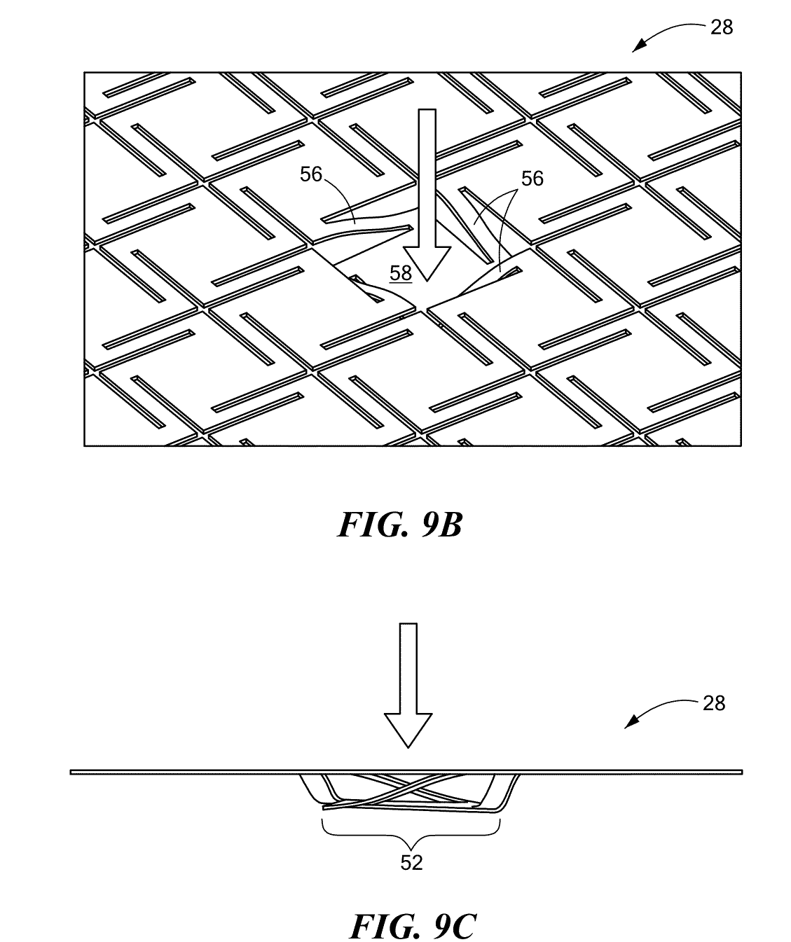

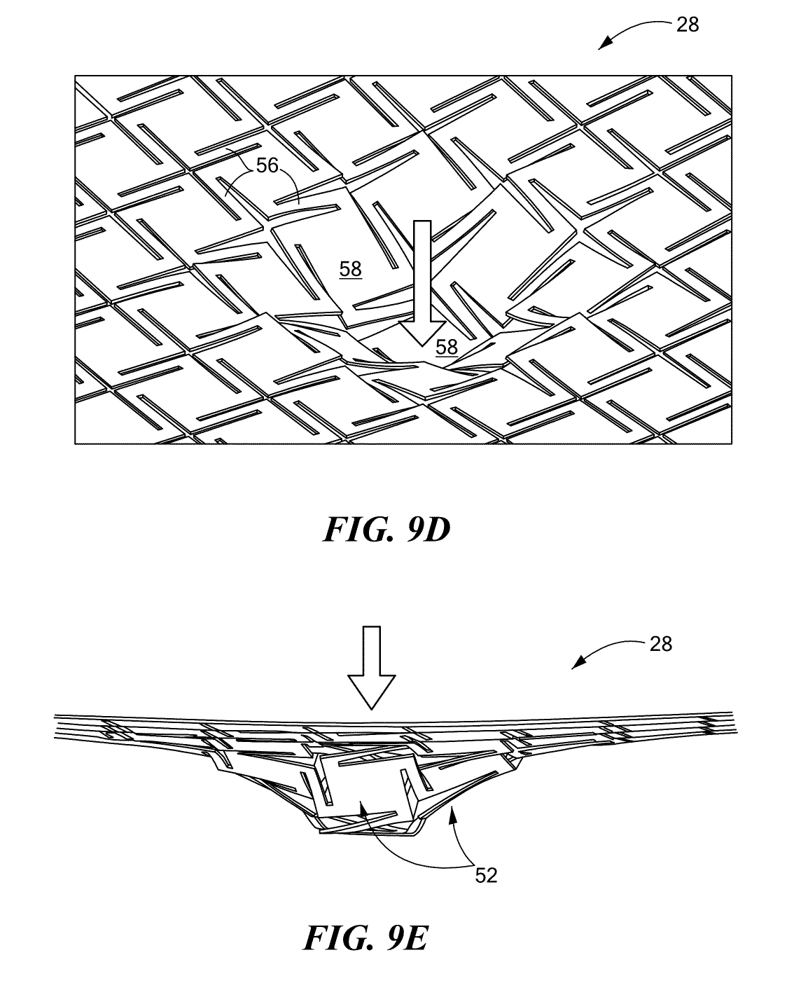

FIGS. 9A through 9E demonstrate the flexibility and effective elasticity of the substrate 28. Specifically, FIG. 9A schematically shows a perspective view of the substrate 28 discussed above with regard to FIG. 7A. At FIGS. 9B and 9C, a focused force is applied to the sensor region 58 of a single substrate segment 52. This force may be applied substantially normal to the face of the substrate 28, or at an angle to the substrate 28.

As shown, the single substrate segment 52 flexes downwardly in a direction having a vector determined by the direction of the force and the orientation of its connectors/springs 56. More specifically, the single substrate segment 52 is configured so it can move relative to other substrate segments 52 when subjected to the single point force. Some other neighboring substrate segments 52 may move to a lesser extent in a generally similar or different direction. As noted above, the substrate segment 52 moves without damaging the circuit traces 60. When the force is released, the substrate segment 52 should substantially return to its original position, favorably causing the substrate 28 to exhibit elastic properties. Accordingly, despite the fact that it includes an inelastic substrate material, the substrate 28 exhibits elastic properties while maintaining the structural integrity of the circuit traces 60.

FIGS. 9D and 9E similarly show a larger point force applied to the same substrate segment 52. In that case, more substrate segments 52 move downwardly with the single substrate segment 52. As with the example of FIGS. 9C and 9D, when the force is released, the substrate segment 52 should substantially return to its original position, favorably causing the substrate 28 to exhibit elastic properties without damaging the circuit traces 60 bridged across the connectors 56. In actual use, a wider force corresponding to the shape of the foot 10 will be applied, causing a similar but more widespread elastic substrate response.

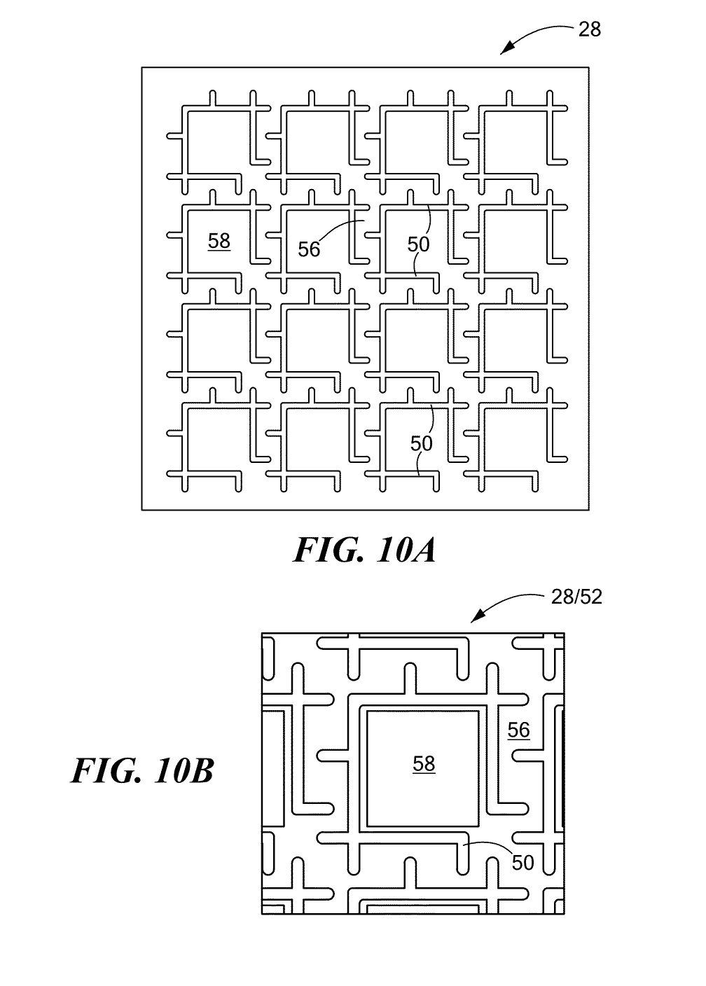

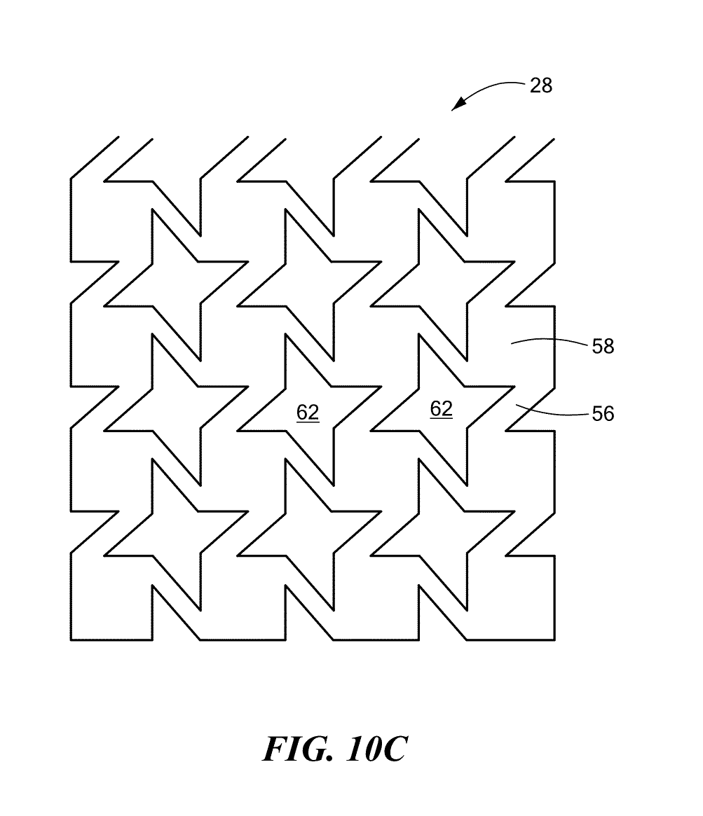

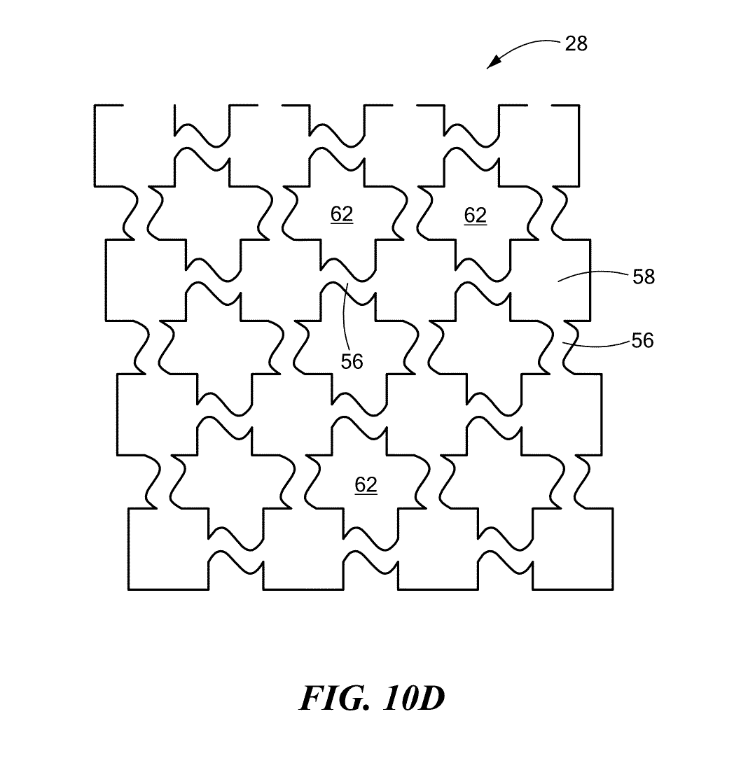

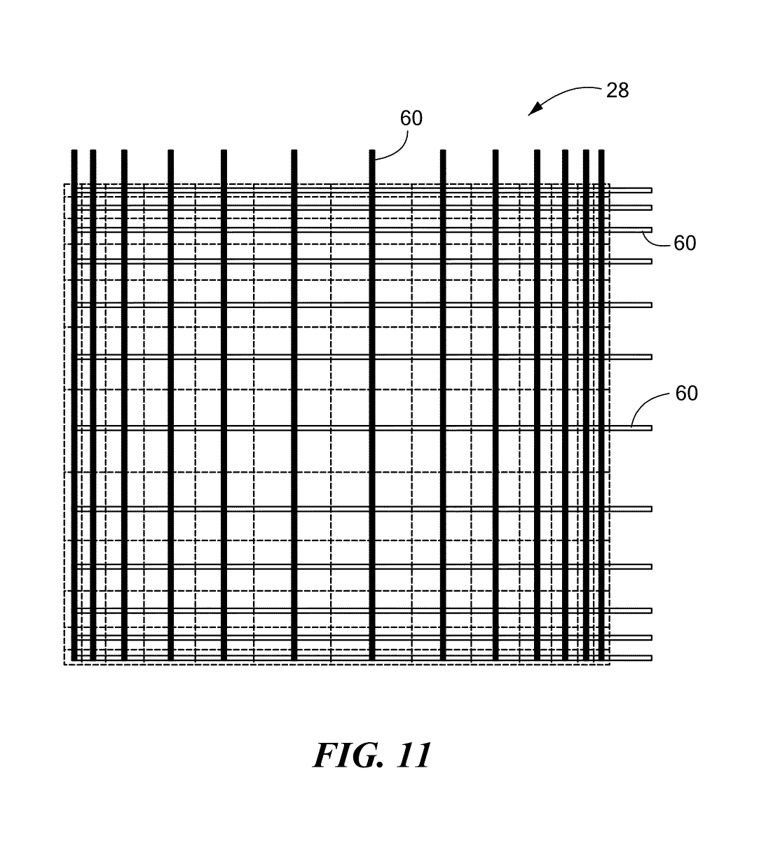

Indeed, alternative embodiments have other arrangements and shapes for the substrate segments 52, including their connectors 56 and sensor regions 58. FIGS. 10A through 10D show a number of other such arrangements. Specifically, FIG. 10A, and its close-up view of one segment in FIG. 10B, schematically shows a generally square sensor region 58 and a convoluted connector 56. Unlike the embodiment of FIG. 6A, the connector 56 in this embodiment has relatively complex geometry to effectively turn in different directions, forming a serpentine shape—it does not have a straight (non-serpentine) shape. As known by those skilled in the art, a serpentine shaped connector 56 may be configured to provide a more controlled spring force due at least to its longer length (if it were straightened out).

FIGS. 10C and 10D schematically shows another substrate configuration with holes 62 in the substrate 28 that form the connectors 56 and sensor regions 58. Those skilled in the art can adjust the shape and size of the holes 62 to provide the appropriate aggregate elasticity and flexibility. It should be noted that the shapes in the figures are but examples and not intended to limit various embodiments of the invention. Those skilled in the art can use any reasonable shape for accomplishing the noted goals. Moreover, like other figures and patents, the substrate 28 and its holes 62 and connectors 56 of FIGS. 10A-10D are not drawn to scale. Instead, certain features are drawn larger to simplify the understanding of certain embodiments.

The prior noted figures generally showed the array of temperature sensors 26 and sensor regions 58 as being generally equally spaced apart. During experimentation, however, the inventors discovered that performance can be improved when the sensors 26 and sensor regions 58 are not necessarily equally spaced apart. FIG. 11 schematically shows a plan view of one embodiment in which the central region of the substrate 28 has a lower density of sensors 26 than the right and left sides (from the perspective of the drawing). In fact, in this embodiment, the right side has a higher density of sensors 26 than that of the left side. These sensors 26 thus are variably spaced apart. The pattern of the matrix of traces 60 accordingly is arranged based on the sensor positions and connector pattern.

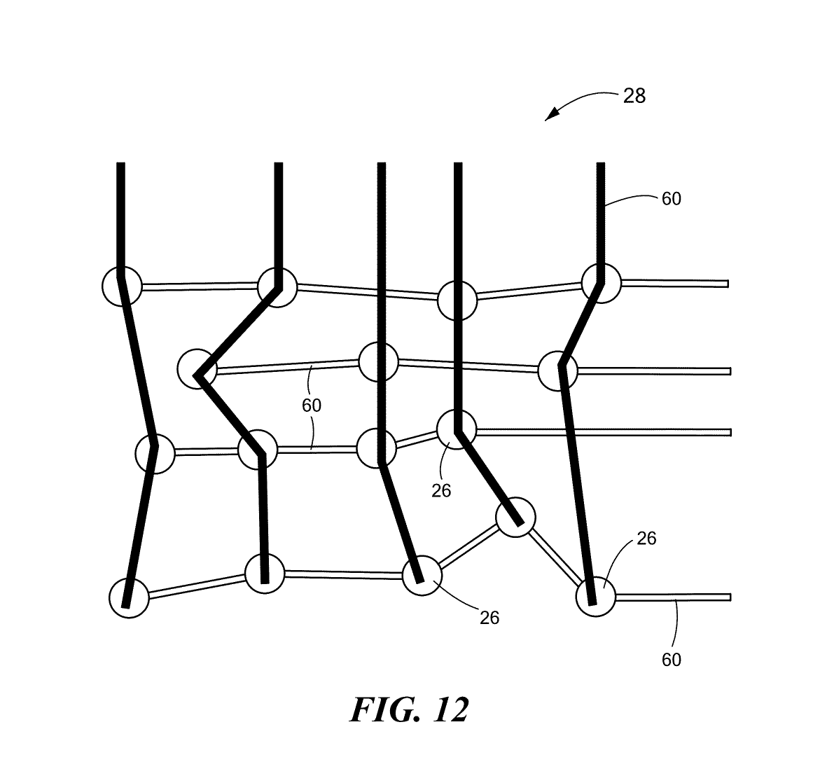

Despite the varying density of the sensors 26 in FIG. 11, its sensors 26 still remain in distinct rows and columns. Some embodiments, however, do not lay out the sensors 26 in organized rows and columns. Other shapes or a random pattern can be used. FIG. 12 shows one such embodiment, in which there are no clearly straight rows and columns. Instead, this embodiment has rows and columns that are not straight and with different numbers of sensors 26. For example, the rightmost column has three sensors 26 while the adjacent column has four sensors 26. In fact, the two rightmost columns share a sensor 26 in this and other embodiments.

More specifically and in some embodiments, pseudo-spectral grids use non-uniform spacing between sensors to improve the accuracy of global interpolation over the grid, which has known computational issues for uniformly-spaced grids. Global interpolation techniques include Chebyshev and Legendre polynomials, which offer super-linear (under many common conditions, exponential) convergence with increasing sensor density. Determining the Chebyshev and Legendre polynomial coefficients for interpolation on a uniform grid is a poorly-posed problem, resulting in Runge phenomenon and inaccurate oscillations when interpolating at the boundaries of the sensor domain; this problem is remedied using non-uniform spectral grids, which cluster points at the edge of the domain, allowing efficient and stable estimation of high-order polynomial coefficients and interpolation using Chebyshev or other global polynomial bases.

Interpolating over a grid of Padua points also allows super-linear and stable interpolation. Unlike spectral grids, which are tensor product grids (i.e., there are discrete rows and columns, and the number of rows in each column is equal and vice versa), Padua interpolation grids do not have aligned rows and columns. They do have minimal growth of interpolating instability with increasing sensor density.

Random grids can also be used to accurately interpolate sensor values using a technique known as “compressive sensing.” If the random grid of sensor values are interpolated with a global basis that is incoherent over the sensor locations and have a sparse representation in that global basis, significantly fewer sensors can be used. One way to understand compressive sensing is as follows: given a signal that can be compressed effectively post-hoc, it is possible to design a grid that collects only the data relevant to the compression, essentially compressing that data as it is collected. Potential bases include but are not limited to wavelets, radial basis functions, and high-order tensor-product polynomials, and bases derived from data collected at high-resolution.

In illustrative embodiments, the sensor array also is configured to substantially mitigate electrical signal bleeding caused by sensors 26 not delivering information to the array output. Without mitigation, the configuration of thermistors with shared row and column conductors effectively creates a network of thermistors. The resistance of the target thermistor at the intersection of the selected column and row conductor is not isolated to the thermistor, but rather is the Thevenin equivalent of the local network of thermistors in which current can leak in the reverse direction through adjacent pathways.

To mitigate that leakage problem, illustrative embodiments have a feedback loop connecting the output voltage to other sensors 26 not being analyzed. Accordingly, the voltage drop across those non-targeted sensors 26 is approximately zero, causing them to produce no leaking current (or a negligible amount of current). Without this leaking current, the target sensor 26 should produce more accurate and substantially sharp, precise output data.

To that end, illustrative embodiments isolate individual resistive sensors 26 arranged in a matrix with shared column and row conductors. In fact, those embodiments provide this arrangement without requiring multiple amplifiers for each output conductor, thus eliminating current leakage through diverging pathways. For example, instead of maintaining the non-energized row conductors at a constant voltage, all of the non-energized columns and rows are maintained at the same voltage as the energized output row conductor. This is accomplished through a feedback loop with unity gain buffer that dynamically clamps the voltage to the energized output. Analog switches at the collection of each of the columns allow the conductor to be temporarily connected to either the energizing voltage supply or the feedback voltage, and at each of the rows to temporarily connect the conductor to either the output amplifier or the feedback voltage.

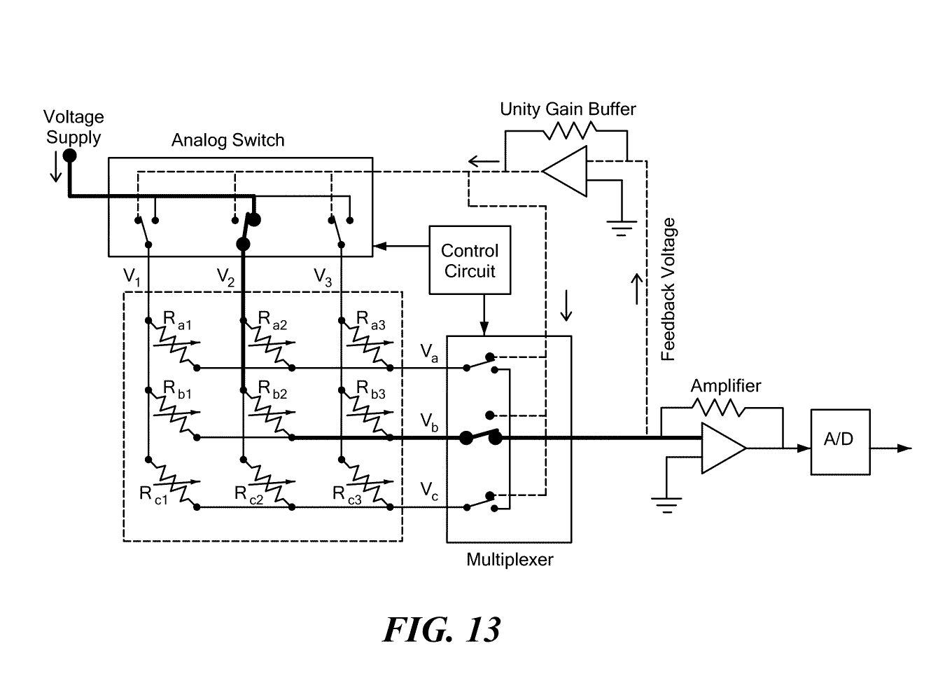

FIG. 13 schematically shows a 3 by 3 sensor array that may implement illustrative embodiments of the invention. Of course, those skilled in the art should understand that principals of this example apply to other types of resistive and non-resistive sensors 26 having different sizes. For example, this arrangement may apply to a 10 by 10 array, or a 5 by 10 array.

The sensor array of FIG. 13 includes a plurality of resistive sensors 26 arranged in an array, and a control system for delivering output from each sensor 26 to a common output. The control system includes a multiplexer and control circuit that cooperate to selectively connect one sensor 26 to a common digital output. As shown, the multiplexer may include a plurality of switches that select one row and one column, thus selecting the sensor 26 at the intersection of the two closed switches. In addition, to mitigate current leakage from other sensors 26, illustrative embodiments also have a feedback loop that connects the output to another multiplexer (via a unity gain buffer), which selectively connects with each column and row of sensors 26.

To select sensor Rb2 for reading, for instance, the control circuit sets the column switches/multiplexers to connect column 2 to the energizing voltage supply, and columns 1 and 3 to the feedback voltage. In addition, the control circuit sets the row switches/multiplexers to connect row b to the output amplifier, and rows a and c to the feedback voltage. In this way, regardless of which sensor 26 is selected, all non-energized pathways are maintained at the same voltage as the output.

With no voltage difference between the conductors, there can be no (or a negligible amount of) current flowing through the resistive sensors 26, favorably isolating the target sensor 26, i.e., there are no diverging branches to reduce the Thevenin Equivalent resistance of the circuit. Furthermore, the only pathway through which current may leak is that having sensors 26 coupled to the energized column (Ra2 and Rc2 in this example) from the energized column to the feedback voltage-maintained rows. In this example, the leakage current passes through only two sensors 26, compared to eight if the non-energized conductors are maintained at a constant voltage. In a larger sensor matrix, this leakage is substantially reduced/mitigated compared to previously designed systems, favorably reducing power consumption.

Illustrative embodiments create a flexible design for a circuit designer. For example, the reduced number of amplifiers in this circuit allows the designer to use fewer components 1) to reduce cost and/or 2) to select a higher precision amplifier, which can improve performance.

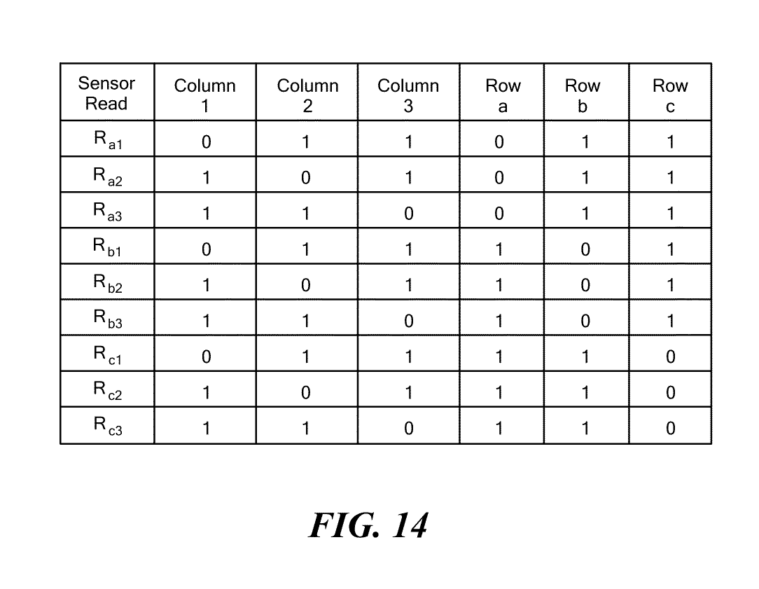

The control logic is straightforward in any size of sensor array. The table of FIG. 14 provides an example for a 3 by 3 element matrix, such as that shown in FIG. 13. For the column switches, 0 signifies connection to the energizing supply voltage and 1 signifies connection to the feedback voltage. For the row switches, 0 signifies connection to the output amplifier and 1 signifies connection to the feedback voltage. This logic can be easily extended for larger arrays.

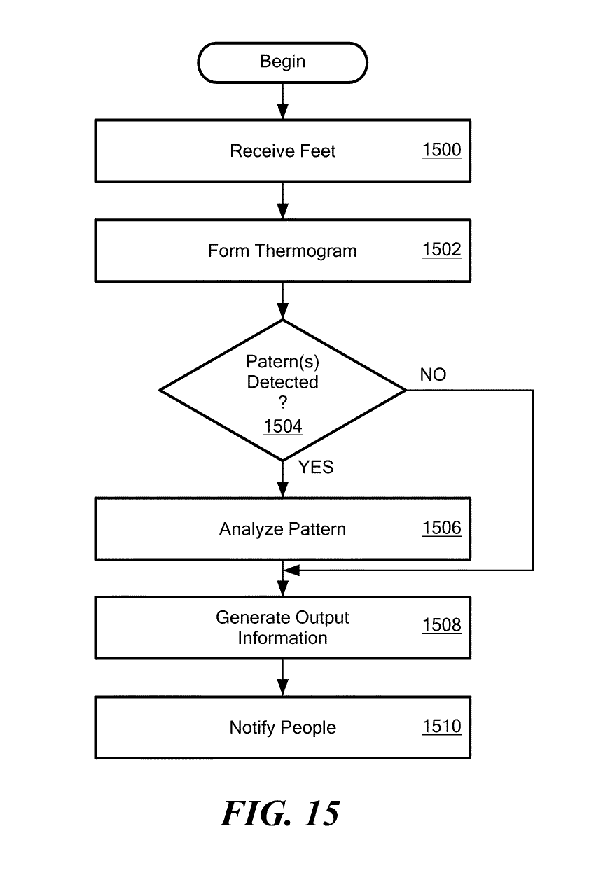

FIG. 15 shows a process to determine the health of the patient's foot 10. Logic within the platform 16, exterior to the platform 16, or spread interior and exterior to the platform 16 may perform these steps. It should be noted that this process is a simplified, high level summary of a much larger process and thus, should not be construed to suggest that only these steps are required. In addition, some of the steps may be performed in a different order than those described below.

The process begins at step 1500, in which the platform 16 receives the patient's feet 10 on its top surface, which may be considered a foot receiving area. For example, as shown in FIG. 2A, the patient may step on the open platform 16 in front of the bathroom sink while washing her hands, brushing her teeth, or performing some other routine, frequent daily task. Presumably, the platform 16 is energized before the patient steps onto it. Some embodiments, however, may require that the platform 16 be affirmatively energized by the patient turning on power in some manner (e.g., actuating a power switch). Other embodiments, however, normally may operate in a low power, conservation mode (a “sleep mode”) that rapidly turns on in response to a stimulus, such as receipt of the patient's feet 10.

Accordingly, the platform 16 controls the sensor array to measure the temperature at the prescribed portions of the patient's foot/sole. At the same time, the user indicator display 18 may deliver affirmative feedback to the patient by any of the above discussed ways. After the patient steps on the platform 16, the temperature sensors 26 may take a relatively long time to ultimately make their readings. For example, this process can take between 30 to 60 seconds. Many people, however, do not have that kind of patience and thus, may step off the platform 16 before it has completed its analysis. This undesirably can lead to inaccurate readings. In addition, these seemingly long delay times can reduce compliance.

The inventors recognized these problems. Accordingly, illustrative embodiments of the invention do not require such long data acquisition periods. Instead, the system can use conventional techniques to extrapolate a smaller amount of real temperature data (e.g., a sparer set of the temperature data) to arrive at an approximation of the final temperature at each point of the foot 10. For example, this embodiment may use techniques similar to those used in high speed thermometers to extrapolate the final temperature data using only one to three seconds of actual temperature data.

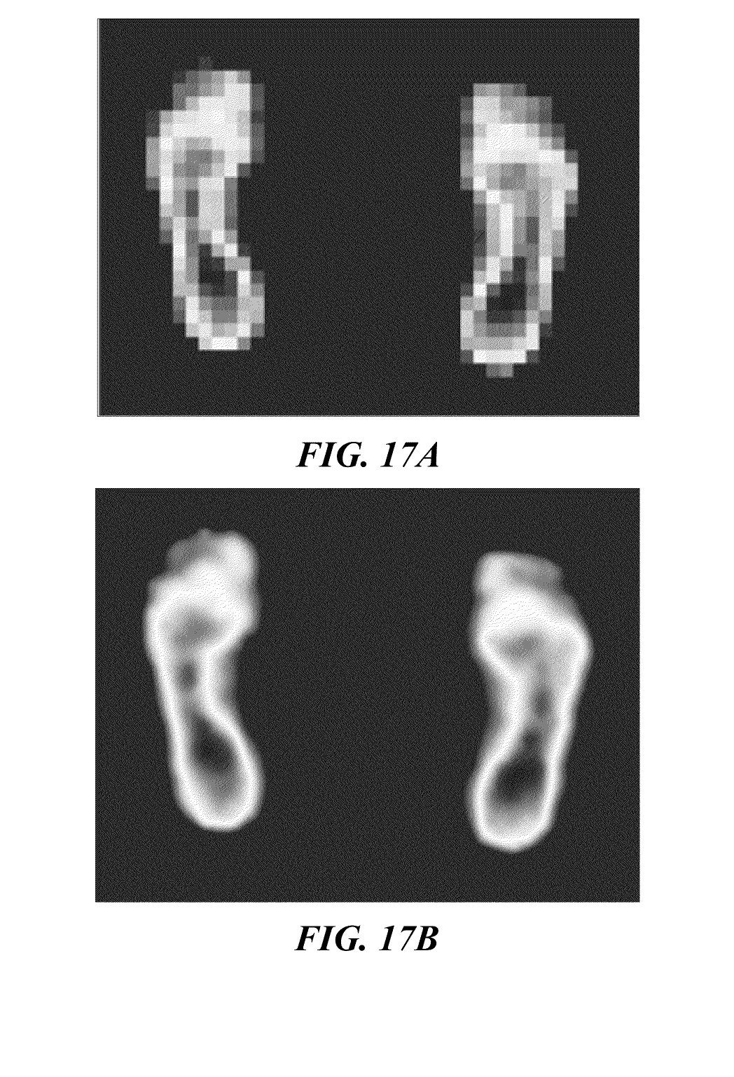

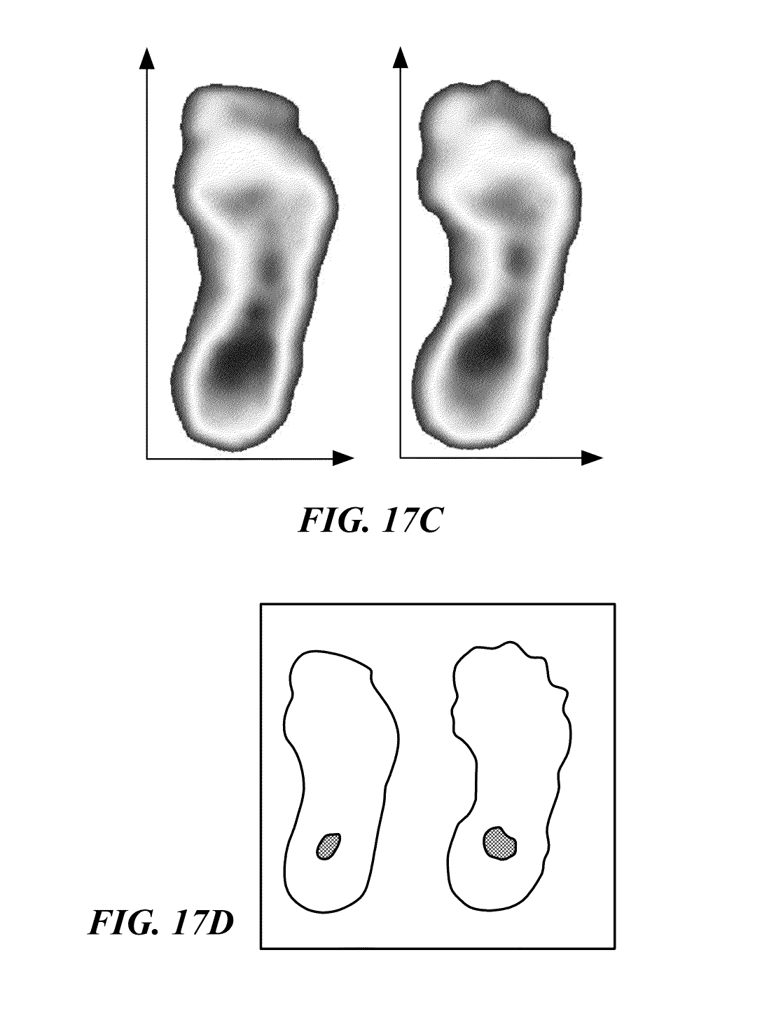

This step therefore produces a matrix of discrete temperature values across the foot 10 or feet 10. FIG. 17A graphically shows one example of this discrete temperature data for two feet 10. As discrete temperature values, this representation does not have temperature information for the regions of the foot 10 between the temperature sensors 26. Accordingly, using this discrete temperature data as shown in FIG. 17A, the process subsequently forms a thermogram of the foot 10 or feet 10 under examination (step 1502).

In simple terms, as known by those in the art, a thermogram is a data record made by a thermograph, or a visual display of that data record. A thermograph simply is an instrument that records temperatures (i.e., the platform 16). As applied to illustrative embodiments, a thermograph measures temperatures and generates a thermogram, which is data, or a visual representation of that data, of the continuous two-dimensional temperature data across some physical region, such as a foot 10. Accordingly, unlike an isothermal representation of temperature data, a thermogram provides a complete, continuous data set/map of the temperatures across an entire two-dimensional region/geography. More specifically, in various embodiments, a thermogram shows (within accepted tolerances) substantially complete and continuous two-dimensional spatial temperature variations and gradients across portions of the sole of (at least) a single foot 10, or across the entire sole of the single foot 10.

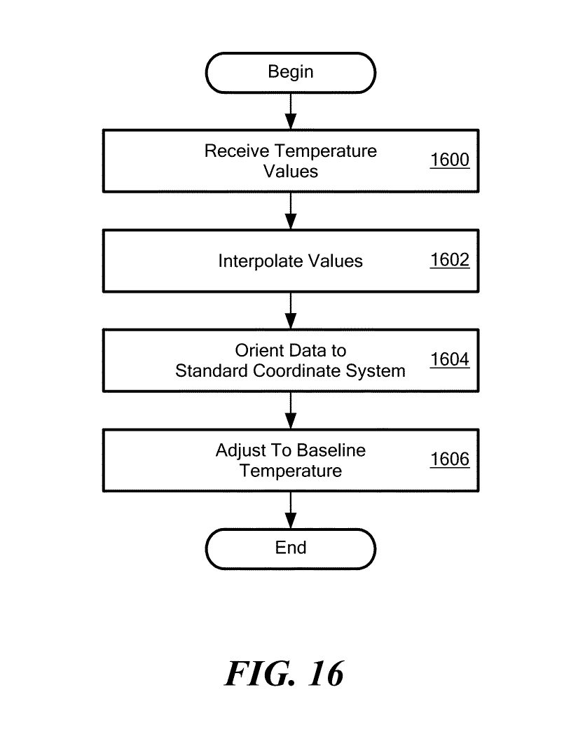

Momentarily turning away from FIG. 15, FIG. 16 shows a process that step 1502 uses to form a thermogram. This discussion will return to FIG. 15 and proceed from step 1502 after completing the discussion of the thermogram formation process of FIG. 16. It should be noted that, in a manner similar to FIG. 15, the process of FIG. 16 is a simplified, high level summary of a larger process and thus, should not be construed to suggest that only these steps are required. In addition, some of the steps may be performed in a different order than those described below.

The process of forming a thermogram begins at step 1600, in which a thermogram generator (not shown) of an analysis engine (not shown) receives the plurality of temperature values, which, as noted above, are graphically shown by FIG. 17A. Of course, the thermogram generator typically receives those temperature values as raw data. The depiction in FIG. 17A therefore is simply for illustration purposes only.

After receiving the temperature values, the process begins calculating the temperatures between the temperature sensors 26. To that end, the process uses conventional interpolation techniques to interpolate the temperature values in a manner that produces a thermogram as noted above (step 1602). Accordingly, for a thermogram of a planar thermodynamic system at steady state, the process may be considered to increase the spatial resolution of the data.

Among other ways, some embodiments may use Laplace interpolation between the temperatures observed at each temperature sensor 26. Laplace interpolation is appropriate for this function given its physical relevance—the heat equation should simplify to the Laplace equation under the assumption of steady state. The interpolant may be constructed by applying a second-order discrete finite difference Laplacian operator to the data, imposing equality conditions on the known temperatures at the sensors 26, and solving the resulting sparse linear system using an iterative solver, such as GMRES.

FIG. 17B schematically shows one example of the thermogram at this stage of the process. This figure should be contrasted with FIG. 17A, which shows a more discrete illustration of the soles of the feet 10.

At this point, the process is considered to have formed the thermogram. For effective use, however, it nevertheless still may require further processing. Step 1604 therefore orients the data/thermogram to a standard coordinate system. To that end, the process may determine the location of the sole of each foot 10, and then transform it into a standard coordinate system for comparison against other temperature measurements on the same foot 10, and on the other foot 10. This ensures that each portion of the foot 10 may be compared to itself from an earlier thermogram. FIG. 17C schematically shows one example of how this step may reorient the thermogram of FIG. 17B.

The position and orientation of the foot 10 on the platform 16 therefore is important when performing this step. For example, to determine the position and orientation of the foot 10, the analysis engine and its thermogram generator simply may contrast the regions of elevated temperature on the platform 16 (i.e., due to foot contact) with those at ambient temperature. Other embodiments may use pressure sensors to form a pressure map of the foot 10.

The process may end at this point, or continue to step 1606, to better contrast warmer portions of the foot 10 against other portions of the foot 10. FIG. 17D schematically shows a thermogram produced in this manner from the thermogram of FIG. 17C. This figure more clearly shows two hotspots on the foot 10 than FIG. 17C. To that end, the process determines the baseline or normal temperature of the foot 10 for each location within some tolerance range. The amount to which the actual temperature of a portion of the foot 10 deviates from the baseline temperature of that portion of the foot 10 therefore is used to more readily show hotspots.

For example, if the deviation is negative, the thermogram may have some shade of blue, with a visual scale of faint blues being smaller deviations and richer blues being larger deviations. In a similar manner, positive deviations may be represented by some shade of red, with a visual scale of faint red being smaller deviations and richer reds being larger deviations. Accordingly, and this example, bright red portions of the thermogram readily show hotspots that may require immediate attention. Of course, other embodiments may use other colors or techniques for showing hotspots. Accordingly, discussion of color coding or specific colors is not intended to limit all embodiments.

Now that the thermogram generator has generated the thermogram, with brighter hotspots and in an appropriate orientation, this discussion returns to FIG. 15 to determine if the thermogram presents or shows any of a number of prescribed patterns (step 1504) and then analyzes any detected pattern (step 1506) to determine if there are hotspots. In particular, as noted, an elevated temperature at a particular portion of the foot 10 may be indicative or predictive of the emergence and risk of a pre-ulcer 14 or ulcer 12 in the foot 10. For example, temperature deviations of about 2 degrees C. or about 4 degrees F. in certain contexts can suggest emergence of an ulcer 12 or pre-ulcer 14. Temperature deviations other than about two degrees C. also may be indicative of a pre-ulcer 14 or ulcer 12 and thus, 2 degrees C. and 4 degrees F. are discussed by example only. Accordingly, various embodiments analyze the thermogram to determine if the geography of the foot 10 presents or contains one or more of a set of prescribed patterns indicative of a pre-ulcer 14 or ulcer 12. Such embodiments may analyze the visual representation of the thermograph, or just the data otherwise used to generate and display a thermograph image—without displaying the thermograph.

A prescribed pattern may include a temperature differential over some geography or portion of the foot 10 or feet 10. To that end, various embodiments contemplate different patterns that compare at least a portion of the foot 10 against other foot data. Among other things, those comparisons may include the following:

1. A comparison of the temperature of the same portion/spot of the same foot 10 at different times (i.e., a temporal comparison of the same spot),

2. A comparison of the temperatures of corresponding portions/spots of the patient's two feet 10 at the same time or at different times, and/or

3. A comparison of the temperature of different portions/spots of the same foot 10 at the same time or at different times.

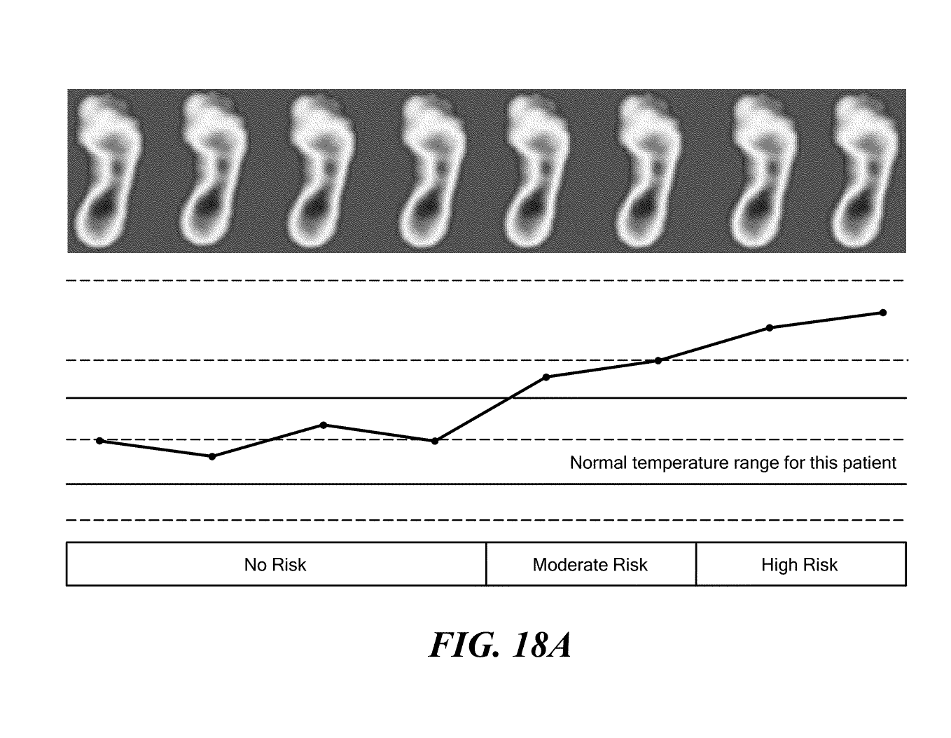

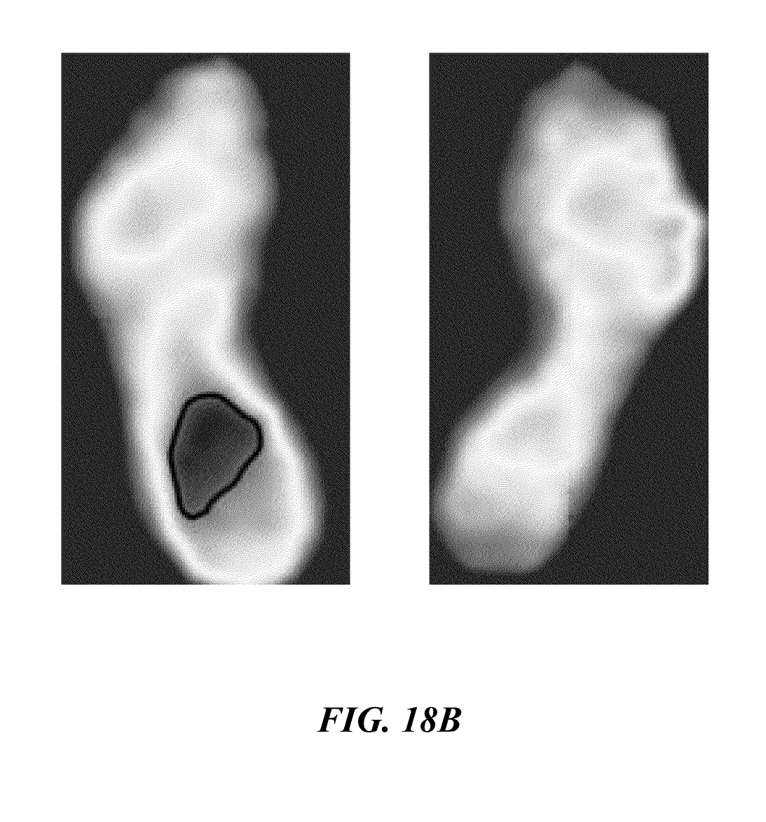

As an example of the first comparison, the pattern may show a certain region of a foot 10 has a temperature that is 4 F higher than the temperature at that same region several days earlier. FIG. 18A schematically shows one example of this, in which a portion of the same foot 10—the patient's left foot 10, has a spot with an increased risk of ulceration.

As an example of the second comparison, the pattern may show that the corresponding portions of the patient's feet 10 have a temperature differential that is 4 degrees F. FIG. 18B schematically shows an example of this, where the region of the foot 10 on the left (the right foot 10) having a black border is hotter than the corresponding region on the foot 10 on the right (the left foot 10).

As an example of the third comparison, the pattern may show localized hotspots and peaks within an otherwise normal foot 10. These peaks may be an indication of pre-ulcer 14 or ulcer 12 emergence, or increased risk of the same, which, like the other examples, alerts caregiver and patient to the need for more vigilance.

Of course, various embodiments may make similar comparisons while analyzing the thermogram for additional patterns. For example, similar to the third comparison, the pattern recognition system (not shown) may have a running average of the temperature of the geography of the entire foot 10 over time. For any particular spot on the foot 10, this running average may have a range between a high temperature and a low temperature. Accordingly, data indicating that the temperature at that given spot is outside of the normal range may be predictive of a pre-ulcer 14 or an ulcer 12 at that location.

Some embodiments may use machine learning and advanced filtering techniques to ascertain risks and predictions, and to make the comparisons. More specifically, advanced statistical models may be applied to estimate the current status and health of the patient's feet 10, and to make predictions about future changes in foot health. State estimation models, such as a switching Kalman filters, can process data as they become available and update their estimate of the current status of the user's feet 10 in real-time. The statistical models can combine both expert knowledge based on clinical experience, and published research (e.g., specifying which variables and factors should be included in the models) with real data gathered and analyzed from users. This permits models to be trained and optimized based on a variety of performance measures.

Models can be continually improved as additional data is gathered, and updated to reflect state-of-the-art clinical research. The models also can be designed to take into account a variety of potentially confounding factors, such as physical activity (e.g., running), environmental conditions (e.g., a cold floor), personal baselines, past injuries, predisposition to developing problems, and problems developing in other regions (e.g., a rise in temperature recorded by a sensor 26 may be due to an ulcer 12 developing in a neighboring region measured by a different sensor 26). In addition to using these models for delivering real-time analysis of users, they also may be used off-line to detect significant patterns in large archives of historical data. For example, a large rise above baseline temperature during a period of inactivity may precede the development of an ulcer 12.

Alternative embodiments may configure the pattern recognition system 68 and analyzer (not shown) to perform other processes that identify risk and emergence, as well as assist in tracking the progressions ulcers 12 and pre-ulcers 14. For example, if there is no ambient temperature data from a thermogram prior to the patient's use of the platform 16, then some embodiments may apply an Otsu filter (or other filter) first to the high resolution thermogram to identify regions with large temperature deviations from ambient. The characteristics of these regions (length, width, mean temperature, etc. . . . ) then may be statistically compared to known distributions of foot characteristics to identify and isolate feet 10. The right foot thermogram may be mirrored and an edge-alignment algorithm can be employed to standardize the data for hotspot identification.

Two conditions can be evaluated independently for hotspot identification. The first condition evaluates to true when a spatially-localized contralateral thermal asymmetry exceeds a pre-determined temperature threshold for a given duration. The second condition evaluates to true when a spatially-localized ipsilateral thermal deviation between temporally successive scans exceeds a pre-determined temperature threshold for a given duration. The appropriate durations and thermal thresholds can be determined from literature review or through application of machine learning techniques to data from observational studies. In the latter case, a support vector machine or another robust classifier can be applied to outcome data from the observational study to determine appropriate temperature thresholds and durations to achieve a desired balance between sensitivity and specificity.

Illustrative embodiments have a set of prescribed patterns against which a pattern recognition system and analyzer compare to determine foot health. Accordingly, discussion of specific techniques above are illustrative of any of a number of different techniques that may be used and thus, are not intended to limit all embodiments of the invention.

The output of this analysis can be processed to produce risk summaries and scores that can be displayed to various users to trigger alerts and suggest the need for intervention. Among other things, state estimation models can simulate potential changes in the user's foot 10 and assess the likelihood of complications in the future. Moreover, these models can be combined with predictive models, such as linear logistic regression models and support vector machines, which can integrate a large volume and variety of current and historical data, including significant patterns discovered during off-line analysis. This may be used to forecast whether the user is likely to develop problems within a given timeframe. The predictions of likelihood can be processed into risk scores, which also can be displayed by both users and other third parties. These scores and displays are discussed in greater detail below.

To those ends, the process continues to step 1508, which generates output information relating to the health of the foot 10. Specifically, at this stage in the process, the analysis engine has generated the relevant data to make a number of conclusions and assessments, in the form of output information, relating to the health of the foot 10. Among other things, those assessments may include the risk of an ulcer 12 emerging anywhere on the foot 10, or at a particular location on the foot 10. This risk may be identified on a scale from no risk to maximum risk.

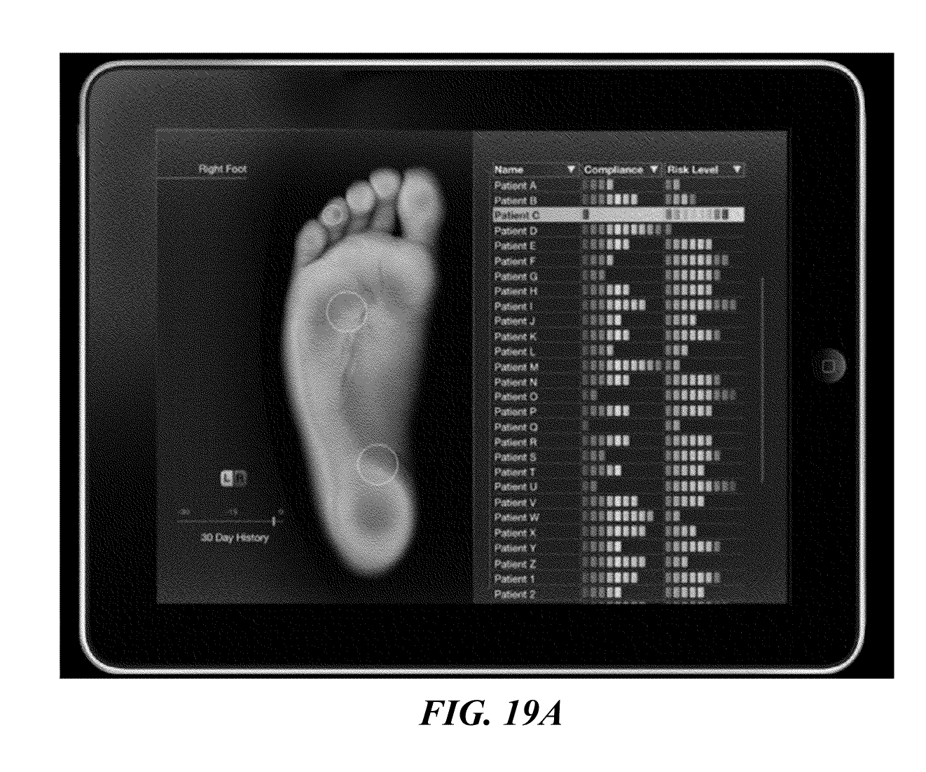

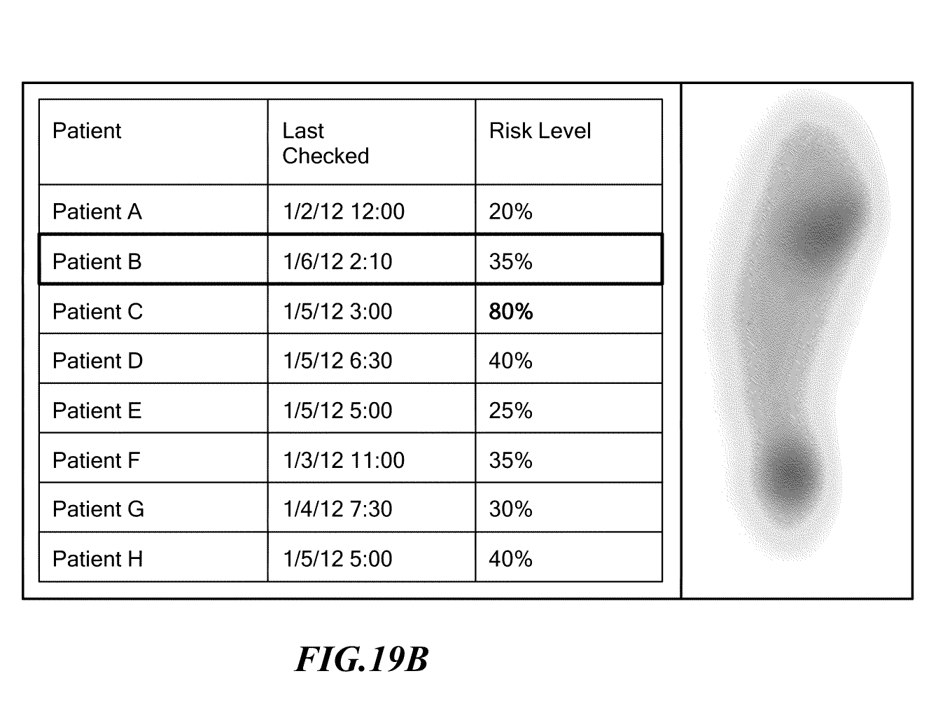

FIG. 19A shows one example of the output information in a visual format with a scale ranking the risk of ulcer emergence. The scale in this example visually displays de-identified patients (i.e., Patient A to Patient 2) as having a certain risk level of developing the foot ulcer 12. The “Risk Level” column shows one way of graphically displaying the output information, in which more rectangles indicate a higher risk of ulcer 12. Specifically, in this example, a single rectangle may indicate minimal or no risk, while rectangles filling the entire length of that table entry may indicate a maximum risk or fully emerged ulcer 12. Selection of a certain patient may produce an image of the foot 10 with a sliding bar showing the history of that patient's foot 10. FIG. 19B schematically shows a similar output table in which the risk level is characterized by a percentage from zero to hundred percent within some time frame (e.g., days). Patient C is bolded in this example due to their 80 percent risk of the emergence of an ulcer 12.

The output table thus may provide the caregiver or healthcare provider with information, such as the fact that Patient B has a 90 percent probability that he/she will develop a foot ulcer 12 in the next 4-5 days. To assist in making clinical treatment decisions, the clinician also may access the patient's history file to view the raw data.

Other embodiments produce output information indicating the emergence of a pre-ulcer 14 at some spot on the foot 10. As known by those skilled in the art, a pre-ulcer 14 may be considered to be formed when tissue in the foot 10 is no longer normal, but it has not ruptured the top layer of skin. Accordingly, a pre-ulcer 14 is internal to the foot 10. More specifically, tissue in a specific region of the foot 10 may not be receiving adequate blood supply and thus, may need more blood. When it does not receive an adequate supply of blood, it may become inflamed and subsequently, become necrotic (i.e., death of the tissue). This creates a weakness or tenderness in that region of the foot 10. Accordingly, a callous or some event may accelerate a breakdown of the tissue, which ultimately may rupture the pre-ulcer 14 to form an ulcer 12.

Illustrative embodiments may detect the emergence of a pre-ulcer 14 in any of a number of manners described above. For example, the system may compare temperature readings to those of prior thermograms, such as the running average of the temperature at a given location. This comparison may show an elevated temperature at that spot, thus signaling the emergence of a new pre-ulcer 14. In more extreme cases, this may indicate the actual emergence of a new ulcer 12.

The emergence or detection of a pre-ulcer 14 can trigger a number of other preventative treatments that may eliminate or significantly reduce the likelihood of the ultimate emergence of an ulcer 12. To that end, after learning about a pre-ulcer 14, some embodiments monitor the progression of the pre-ulcer 14. Preferably, the pre-ulcer 14 is monitored during treatment in an effort to heal the area, thus avoiding the emergence of an ulcer 12. For example, the caregiver may compare each day's thermogram to prior thermograms, thus analyzing the most up to date state of the pre-ulcer 14. In favorable circumstances, during a treatment regimen, this comparison/monitoring shows a continuous improvement of the pre-ulcer 14, indicating that the pre-ulcer 14 is healing. The output information therefore can have current and/or past data relating to the pre-ulcer 14, and the risk that it poses for the emergence of an ulcer 12.

Sometimes, patients may not even realize that they have an ulcer 12 until it has become seriously infected. For example, if the patient undesirably does not use the foot monitoring system for a long time, he/she may already have developed an ulcer 12. The patient therefore may step on the platform 16 and the platform 16 may produce output information indicating the emergence of an ulcer 12. To that end, the analyzer may have prior baseline thermogram (i.e., data) relating to this patient's foot 10 (showing no ulcer 12), and make a comparison against that baseline data to determine the emergence of an actual ulcer 12. In cases where the data is questionable about whether it is an ulcer 12 or a pre-ulcer 14, the caregiver and/or patient nevertheless may be notified of the higher risk region of the foot 10 which, upon even a cursory visual inspection, should immediately reveal the emergence of an ulcer 12.

The process concludes at step 1510, in which the process (optionally) manually or automatically notifies the relevant people about the health of the foot 10. These notifications or messages (a type of “risk message”) may be in any of a number of forms, such as a telephone call, a text message, e-mail, and data transmission, or other similar mechanism. For example, the system may forward an e-mail to a healthcare provider indicating that the right foot 10 of the patient is generally healthy, while the left foot 10 has a 20 percent risk of developing an ulcer 12, and a pre-ulcer 14 also has emerged on a specified region. Armed with this information, the healthcare provider may take appropriate action, such as by directing the patient to stay off their feet 10, use specialized footwear, soak their feet 10, or immediately check into a hospital.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product (or in a computer process) for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium.

The medium may be either a tangible medium (e.g., optical or analog communications lines) or a medium implemented with wireless techniques (e.g., WIFI, microwave, infrared or other transmission techniques). The medium also may be a non-transient medium. The series of computer instructions can embody all or part of the functionality previously described herein with respect to the system. The processes described herein are merely exemplary and it is understood that various alternatives, mathematical equivalents, or derivations thereof fall within the scope of the present invention.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the larger network 44 (e.g., the Internet or World Wide Web). Of course, some embodiments of the invention may be implemented as a combination of both software (e.g., a computer program product) and hardware. Still other embodiments of the invention are implemented as entirely hardware, or entirely software.

Although the above discussion discloses various exemplary embodiments of the invention, it should be apparent that those skilled in the art can make various modifications that will achieve some of the advantages of the invention without departing from the true scope of the invention.

What is claimed is:

1. An apparatus for measuring the temperature distribution over the sole of the foot, the apparatus comprising:
a flexible substrate formed at least in part from a substrate material configured to be bendable without breaking when receiving the sole of a human foot, the substrate defining a plurality of discontinuities forming a plurality of substrate segments, each substrate segment comprising a sensor region having a surface for coupling a sensor and at least one connector to connect to at least one sensor region of an adjacent substrate segment, each of a plurality of the sensor regions being configured to be movable relative to other sensor regions when subjected to a mechanical force applied to its surface, the discontinuities causing the flexible substrate to exhibit elastic properties in the aggregate so that it can recover its size and shape after deformation along one or more of the discontinuities;
a plurality of resistive temperature sensors coupled with the flexible substrate, the plurality of resistive temperature sensors being contact sensors for measuring the temperature of a human foot in thermal contact with it, a plurality of the sensor regions of the substrate segments each coupled with at least one of the plurality of temperature sensors; and
a matrix of circuit conductors extending across the flexible substrate to electrically interconnect the plurality of resistive contact temperature sensors, a plurality of the connectors each physically bridging a portion of the matrix of conductors between sensor regions of adjacent substrate segments to electrically connect adjacent temperature sensors,
at least some of the substrate segments and matrix of circuit conductors being configured to permit relative substrate segment movement without mechanically breaking the portion of the matrix of circuit conductors on the connector between substrate segments moving relatively to each other.

2. The apparatus as defined by claim 1 wherein the flexible substrate is formed at least in part from an inelastic substrate material.

3. The apparatus as defined by claim 1 wherein the flexible substrate is formed at least in part from an elastic substrate material.

4. The apparatus as defined by claim 1 wherein the substrate forms an open platform.

5. The apparatus as defined by claim 1 wherein the substrate forms a closed platform.

6. The apparatus as defined by claim 1 wherein each of a plurality of the connectors has a serpentine shape.

7. The apparatus as defined by claim 1 wherein a plurality of the discontinuities forms an opening between adjacent substrate segments.

8. The apparatus as defined by claim 1 wherein at least two adjacent substrate segments abut each other.

9. The apparatus as defined by claim 1 wherein the substrate material comprises circuit board material.

10. The apparatus as defined by claim 9 wherein the substrate comprises a flexible circuit or polymer.

11. The apparatus as defined by claim 1 wherein each of a plurality of the sensor regions are configured to be movable relative to other sensor regions when subjected to a mechanical force applied generally normal to its surface.

12. The apparatus as defined by claim 1 wherein the plurality of resistive temperature sensors is non-uniformly spaced across the flexible substrate.

13. The apparatus as defined by claim 1 further comprising logic for determining either the risk of an ulcer forming on the foot, or the presence of an ulcer on the foot.

14. The apparatus as defined by claim 1 further comprising logic for measuring the temperature distribution across the foot and forming a thermogram of the temperature distribution.

15. An apparatus for analyzing the temperature distribution over the sole of the human foot, the apparatus comprising:
an open platform comprising a flexible substrate formed at least in part from a substrate material configured to be bendable without breaking when receiving the sole of a human foot, the substrate material being inelastic, the substrate forming a plurality of substrate segments, each substrate segment comprising a sensor region for coupling a sensor and at least one connector to connect to at least one sensor region of an adjacent substrate segment, each of a plurality of the sensor regions being configured to be movable relative to other sensor regions when subjected to a mechanical force applied to its surface, the discontinuities causing the flexible substrate to exhibit elastic properties so that it can recover its size and shape after deformation along one or more of the discontinuities;
a plurality of resistive temperature sensors coupled with the flexible substrate, a plurality of the sensor regions of the substrate segments each coupled with at least one of the plurality of temperature sensors; and
circuit conductors extending across the flexible substrate to electrically interconnect the plurality of resistive temperature sensors, a plurality of the connectors each physically bridging a portion of the conductors between sensor regions of adjacent substrate segments to electrically connect adjacent temperature sensors,
at least some of the substrate segments and circuit conductors being configured to permit relative substrate segment movement without mechanically breaking the portion of the circuit conductors on the connector between substrate segments moving relatively to each other.

16. The apparatus as defined by claim 15 wherein the plurality of resistive temperature sensors is non-uniformly spaced across the flexible substrate.

17. The apparatus as defined by claim 15 wherein the flexible substrate is formed at least in part from an elastic substrate material.

18. The apparatus as defined by claim 15 wherein each of a plurality of the connectors has a serpentine shape.

19. The apparatus as defined by claim 15 wherein a plurality of the discontinuities forms an opening between adjacent substrate segments.

20. The apparatus as defined by claim 15 wherein at least two adjacent substrate segments abut each other.

21. The apparatus as defined by claim 15 wherein the substrate material comprises circuit board material.

22. The apparatus as defined by claim 21 wherein the substrate comprises a flexible circuit or FR-4.

23. The apparatus as defined by claim 15 wherein each of a plurality of the sensor regions are configured to be movable relative to other sensor regions when subjected to a mechanical force applied generally normal to its surface.

24. The apparatus as defined by claim 15 further comprising logic for determining either the risk of an ulcer forming on the foot, or the presence of an ulcer on the foot.

25. The apparatus as defined by claim 15 further comprising logic for measuring the temperature distribution across the foot and forming a thermogram of the temperature distribution.

26. The apparatus as defined by claim 15 wherein the substrate is generally conformable to the sole of the human foot.

27. The apparatus as defined by claim 15 wherein the plurality of resistive temperature sensors form a matrix with a varying sensor density.

28. The apparatus as defined by claim 15 wherein the plurality of resistive sensors are variably spaced apart.

29. The apparatus as defined by claim 15 further comprising:
an output;
a multiplexer configured to selectively connect at least one selected sensor with the output; and
a feedback loop configured to selectively connect the output with unselected sensors in the plurality of sensors.
